(19) United States
(12) Patent Application Publication
(10) Pub. No.: US 2015/0030562 A1
(43) Pub. Date: Jan. 29, 2015

(54) METHODS OF TREATING OR PREVENTING VIRAL DISEASES BY BLOCKING INTERLEUKIN-21

(71) Applicant: The United States of America, as represented by Secretary, Department of Health and Human Services, Bethesda, MD (US)

(72) Inventors: Warren J. Leonard, Bethesda, MD (US); Rosanne Spolski, Silver Spring, MD (US)

(73) Assignee: The United States of America, as represented by the Secretary, Dept. of Health and Human Services, Bethesda, MD (US)

(21) Appl. No.: 14/367,313

(22) PCT Filed: Dec. 21, 2012
(86) PCT No.: PCT/US2012/071173

§ 371 Date: Jun. 20, 2014

Related U.S. Application Data
(60) Provisional application No. 61/579,801, filed on Dec. 23, 2011.

(51) Int. Cl. C07K 14/715; A61K 38/20; C07K 16/28; C07K 14/54; C12N 15/113; C07K 16/24

(52) U.S. Cl. CPC C07K 14/7155; C12N 15/113; C07K 16/244; C07K 16/2863; C07K 14/54; A61K 38/20; C07K 2319/30; A61K 2039/505
USPC 424/85.2; 514/44 A; 424/133.1; 424/158.1; 424/172.1

(57) ABSTRACT

The invention provides a method of treating or preventing viral diseases in a mammal comprising administering to the mammal an interleukin (IL)-21 blocking agent in an amount effective to treat or prevent the viral disease in the mammal. Also provided is a method of reducing the activation or recruitment of immune cells in a mammal comprising administering to the mammal an IL-21 blocking agent in an amount effective to reduce the activation or recruitment of immune cells in the mammal. Methods of decreasing the expression of at least one cytokine or at least one protein in a mammal comprising administering to the mammal an IL-21 blocking agent in an amount effective to decrease the expression of the cytokine or the protein are also provided.

### CROSS-REFERENCE TO RELATED APPLICATION

This patent application claims the benefit of U.S. Provisional Patent Application No. 61/579,801, filed Dec. 23, 2011, which is incorporated by reference in its entirety herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 51,862 Byte ASCII (Text) file named “711840ST25.TXT,” dated Dec. 6, 2012.

### BACKGROUND OF THE INVENTION

There are few effective treatments for many viral diseases including, for example, viral pneumonia. Reduction of the disease's severity, complications, the rate of viral transmission, and prevention of viral damage to organs and systems such as the liver, lungs, heart, central nervous system, and the gastrointestinal system, pose challenges to the development of effective treatments for viral diseases. Accordingly, there is a need for improved methods of treating viral diseases.

### BRIEF SUMMARY OF THE INVENTION

An embodiment of the invention provides a method of treating or preventing a viral disease in a mammal comprising administering to the mammal an interleukin (IL)-21 blocking agent in an amount effective to treat or prevent the viral disease in the mammal.

Another embodiment of the invention provides a method of prolonging the survival of a mammal suffering from a viral disease comprising administering to the mammal an interleukin (IL)-21 blocking agent in an amount effective to prolong the survival of the mammal suffering from the viral disease.

Another embodiment of the invention provides a method of reducing the activation or recruitment of immune cells in a mammal comprising administering to the mammal an IL-21 blocking agent in an amount effective to reduce the activation or recruitment of immune cells in the mammal.

Still another embodiment of the invention provides a method of decreasing the expression of at least one cytokine and/or chemokine in a mammal comprising administering to the mammal an IL-21 blocking agent in an amount effective to decrease the expression of the cytokine and/or chemokine, wherein the at least one cytokine and/or chemokine is selected from the group consisting of interferon (IFN)-γ, IL-6, CXCL1, IL-17α, and IL-1β.

Another embodiment of the invention provides a method of decreasing the expression of at least one protein in a mammal comprising administering to the mammal an IL-21 blocking agent in an amount effective to decrease the expression of the protein, wherein the at least one protein is selected from the group consisting of MMP8 and S100A8.

### BRIEF DESCRIPTION OF THE SEVERAL VIEWS OF THE DRAWING(S)

FIGS. 1A and 1B are graphs showing relative expression of (A) pneumonia virus of mice (PVM) SH mRNA and (B) Il21 mRNA in lung of C57BL/6 wild-type (WT) mice inoculated with PVM and sacrificed at indicated time points. Shown are values ±S.E.M. relative to Rp17 expression (n=5 for each time point). Statistical significance is indicated by * P<0.05, ** P<0.01, *** P<0.001.

FIG. 1C is a graph showing the percentage of IL-21⁺/CD4⁺ T cells in the lung or spleen of Il21-mCherry transgenic reporter mice (TG) or WT littermates that were infected with PVM (or not infected with PVM (ctrl)). Lung or spleen cells were isolated 6 days after infection, and IL-21 expression was measured by flow cytometry after surface staining with anti-CD4 and anti-TCRβ. A representative experiment is shown; the experiment was performed three times with similar results. Statistical significance is indicated by ** P<0.01, *** P<0.001.

FIG. 1D is a graph showing the percentage of ICOS/CXCR5 positive CD4⁺ T cells measured in the lung, mediastinal lymph node (MLN) or spleen of Il21-mCherry transgenic reporter mice (mCherry⁺; solid bars) or WT littermates (mCherry^{neg}; hatched bars) A summary of three experiments is shown.

FIGS. 2A-2D are graphs showing the total number of cells in bronchoalveolar lavage (BAL) fluid (A), the total number of cells isolated from one lobe of the lung (B), the number of neutrophils in BAL fluid (C), or the number of neutrophils in lung (D) at the indicated time points after PVM infection of WT or Il21r^{-/-} mice. The total number of neutrophils was calculated after analyzing the percent Ly6G⁺CD11b⁺ cells in either the BAL fluid (C) or lung (D). Shown are the means±S.E.M from one of three experiments with similar results (n=5-8 mice per group). Statistical significance is indicated by * P<0.05, ** P<0.01, *** P<0.001.

FIGS. 2E and 2F are graphs showing relative expression of Mmp8 mRNA (E) or S100a8 mRNA (F) in one lung lobe at the indicated time points after PVM infection of WT (solid bars) or Il21r^{-/-} (hatched bars) mice. Shown are the means±S.E.M from one of three experiments with similar results (n=5-8 mice per group). Statistical significance is indicated by ** P<0.01, *** P<0.001.

FIGS. 3A-3D are graphs showing the total number of CD4⁺ T cells (A), CD8⁺ T cells (B), γδ T cells (C), and NK cells (D) in lungs of WT (solid bars) and Il21r^{-/-} (hatched bars) mice as measured by flow cytometry. Total cells were calculated from the percentage of each population and the total cell number isolated from one lung lobe. Shown are the means±S.E.M. for one of three experiments with similar results (n=5-8 mice per group). Statistical significance is indicated by * P<0.05, ** P<0.01, *** P<0.001.

FIGS. 3E-3F are graphs showing the percentage of IFNγ⁺CD8⁺ T cells (E) and the calculated value of total IFNγ⁺CD8⁺ T cells (F) after interferon (IFN)-γ-producing cells of WT (solid bars) and Il21r^{-/-} (hatched bars) mice were incubated with PMA/ionomycin/GOLGIPLUG protein transport inhibitor for 4 hr after isolation. Shown are the means±S.E.M. for one of three experiments with similar results (n=5-8 mice per group). Statistical significance is indicated by * P<0.05, ** P<0.01.

FIGS. 4A-4F are graphs showing the relative expression of mRNA encoding IL-17A (A), IL-22 (B), TNFα (C), IFNγ (D), IL-1β (E), and IL-6 (F) that was isolated from the lung of WT (solid bars) and Il21r^{-/-} (hatched bars) mice after PVM infection. Relative levels of mRNA were quantitated by reverse transcriptase polymerase chain reaction (RT-PCR). Shown are the means±S.E.M. from one of three experiments with similar results (n=5-8 mice per group). Statistical significance is indicated by * P<0.05, ** P<0.01.

FIGS. 4G-4H are graphs showing IL-6 protein levels (pg/ml) in BAL fluid (G) or lung homogenate (H) of WT and Il21r^{-/-} mice as measured by enzyme-linked immunosorbent assay (ELISA). Shown are the means±S.E.M. from one of three experiments with similar results (n=5-8 mice per group). Statistical significance is indicated by ** P<0.01, *** P<0.001.

FIGS. 5A-5D are graphs showing relative expression of mRNA encoding the chemokines CXCL1 (A), CXCL10 (B), CCL3 (C), and CXCL2 (D) that was isolated from the lung of WT (solid bars) and Il21r^{-/-} (hatched bars) mice after PVM infection. Relative levels of mRNA were quantitated by RT-PCR. Shown are the means±S.E.M. from one of three experiments with similar results (n=5-8 mice per group). Statistical significance is indicated by * P<0.05, ** P<0.01.

FIGS. 5E-5F are graphs showing CXCL1 protein levels (pg/ml) in BAL fluid (E) or in equal amounts (25 μg) of protein from lung homogenates (F) of WT and Il21r^{-/-} mice as measured by ELISA. Shown are the means±S.E.M. from one of three experiments with similar results (n=5-8 mice per group). Statistical significance is indicated by ** P<0.01, *** P<0.001.

FIG. 6A is a graph showing the total number of cells measured by flow cytometry in mediastinal lymph nodes either before or at day 6 after PVM infection of WT and Il21r^{-/-} mice. Shown are pooled data from three independent experiments. Statistical significance is indicated by *** P<0.001.

FIGS. 6B-6C are graphs showing the total number of CD4⁺ T cells (B) or CD8⁺ (C) T cells based on the percentage of each population and the total cell number either before or at day 6 after PVM infection of WT and Il21r^{-/-} mice. Cells were measured by flow cytometry in mediastinal lymph nodes either before or at day 6 after PVM infection. Shown are pooled data from three independent experiments. Statistical significance is indicated by * P<0.05, ** P<0.01.

FIGS. 6D-6H are graphs showing the relative expression of mRNA encoding IFN-γ (D), TNFα (E), IL-17α(F), IL-6 (G), and IL-1β (H) isolated from MLN of WT and Il21r^{-/-} mice either before or at day 6 after PVM infection. Shown are pooled data from 3 independent experiments. Statistical significance is indicated by * P<0.05.

FIGS. 7A-7B are graphs showing the total number of cells measured in BAL fluid (A) and lungs (B) of WT and IL-21 transgenic mice (TG21). Statistical significance is indicated by * P<0.05, *** P<0.001.

FIG. 7C is a graph showing the percentage of neutrophils in BAL fluid and lung of WT and TG21 mice as determined by flow cytometry of Ly6G⁺CD11b⁺ cells. Statistical significance is indicated by * P<0.05, *** P<0.001.

FIGS. 7D-7F are graphs showing relative expression of Cxcl1 (D), Il6 (E) and Mmp8 (F) mRNA isolated from lung of WT and TG21 mice and measured by RT-PCR. Statistical significance is indicated by ** P<0.01.

FIGS. 7G and 7H are graphs showing relative expression of Il6 mRNA measured by RT-PCR (G) and IL-6 protein measured by ELISA (H). Splenic dendritic cells were isolated and stimulated in vitro with IL-21 for 5 h. Il6 mRNA was measured by RT-PCR at 6 hours and IL-6 protein was measured at 16 h. Statistical significance is indicated by ** P<0.01, *** P<0.001.

FIG. 8A is a graph showing the percent survival of WT (solid line) and Il21r^{-/-} (dotted line) mice at days following infection with PVM. Survival of the mice was monitored daily over the next three weeks. Infection of the long-term survivor was documented by confirming sero-conversion to PVM antigens (SMART-M12, El Cerrito, Calif.; data not shown). Statistical significance was evaluated using Kaplan-Meier survival curve (GraphPad Prism). n=19 mice per group.

FIG. 8B is a graph showing relative expression of PVM SH in total lung RNA of WT (solid bars) and Il21r^{-/-} (hatched bars) mice as measured by RT-PCR. Shown are the means+S.E.M. from one of three experiments with similar results (n=5-8 mice per group).

FIGS. 8C and 8E are graphs showing the percent survival of WT mice that received 50 μg of either IL-21R/Fc (dotted line) or control Fc (solid line) intratracheally one day prior to and 2 days post-inoculation with PVM at days following PVM infection. Mice in (C) and (E) received 60 plaque-forming units (pfu) and 12 pfu, respectively, of PVM intranasally. In (C), p=0.001 and in (E) p=0.039, with 10 mice per group in each panel.

FIGS. 8D and 8F are graphs showing the relative expression of PVM SH mRNA of total lung RNA at day 6 after PVM infection of WT mice that received 50 μg of either IL-21R/Fc (hatched bars) or control Fc (solid bars). mRNA expression was measured by RT-PCR. (D) corresponds to the experiment in FIGS. 8C and (F) corresponds to the experiment in FIG. 8E. Shown are the means+S.E.M. from 5 mice in each group.

FIG. 9 is a graph showing the percent survival of WT mice that received 50 μg of either IL-21R/Fc (solid line) or control Fc (dotted line) intranasally on days 3 and 4 post-inoculation with PVM at days following PVM infection.

### DETAILED DESCRIPTION OF THE INVENTION

Interleukin-21 (IL-21) is a pleiotropic, four α-helical bundle type I cytokine that is produced primarily by CD4⁺ T cell populations, including T follicular helper cells, Th17 populations, and NKT cells. IL-21 acts on a broad range of target cells including B cells, T cells, natural killer cells, dendritic cells, macrophages, and epithelial cells. The functional IL-21 receptor (IL-21R) is a heterodimer of an IL-21-specific protein, IL-21R, and the common cytokine receptor γ chain, γ_{c}.

It has been discovered that blocking IL-21 enhances survival following viral infection. Accordingly, an embodiment of the invention provides a method of treating or preventing a viral disease in a mammal comprising administering to the mammal an interleukin (IL)-21 blocking agent in an amount effective to treat or prevent the viral disease in the mammal.

The viral disease may be caused by any virus. In an embodiment of the invention, the viral disease is caused by a virus selected from the group consisting of herpes viruses, pox viruses, hepadnaviruses, papilloma viruses, adenoviruses, coronoviruses, orthomyxoviruses, paramyxoviruses, flaviviruses, and caliciviruses. In a preferred embodiment, the viral disease is caused by a virus selected from the group consisting of pneumonia virus of mice (PVM), respiratory syncytial virus (RSV), influenza virus, herpes simplex virus, Epstein-Barr virus, varicella virus, cytomegalovirus, hepatitis A virus, hepatitis B virus, hepatitis C virus, human T-lymphotropic virus, calicivirus, adenovirus, and Arena virus. Mouse PVM is a negative strand RNA virus that is in the same family (Paramyxoviridae) and genus (Pneumovirus) as human RSV. PVM infection can result in disease in mice similar to the more severe forms of RSV infection in humans.

The viral disease may be any viral disease affecting any part of the body. In an embodiment of the invention, the viral disease is selected from the group consisting of influenza, pneumonia, herpes, hepatitis, hepatitis A, hepatitis B, hepatitis C, chronic fatigue syndrome, sudden acute respiratory syndrome (SARS), gastroenteritis, enteritis, carditis, encephalitis, bronchiolitis, respiratory papillomatosis, meningitis, and mononucleosis. In a preferred embodiment, the viral disease is a pulmonary viral disease. In a particularly preferred embodiment, the pulmonary viral disease is pneumonia.

The pneumonia may be caused by any virus capable of causing pneumonia in a mammal. In an embodiment of the invention, the pneumonia is caused by at least one virus selected from the group consisting of pneumonia virus of mice (PVM), respiratory syncytial virus (RSV), influenza, herpes, and varicella. In a preferred embodiment, the pneumonia is caused by PVM or RSV.

Another embodiment of the invention provides a method of prolonging the survival of a mammal suffering from a viral disease comprising administering to the mammal an IL-21 blocking agent in an amount effective to prolong the survival of the mammal suffering from the viral disease. In this regard, the mammal suffering from the viral disease survives for a longer time period when administered the IL-21 blocking agent as compared to a mammal suffering from the viral disease that is not administered the IL-21 blocking agent. Survival may be prolonged by any period of time, e.g., an hour or more, six hours or more, twelve hours or more, three days or more, seven days or more, a month or more, or six months or more, or a year or more. The viral disease may be any of the viral diseases discussed herein with respect to other aspects of the invention, or may be caused by any of the viruses discussed herein with respect to other aspects of the invention.

Another embodiment of the invention provides a method of reducing the activation or recruitment of immune cells in a mammal comprising administering to the mammal an IL-21 blocking agent in an amount effective to reduce the activation or recruitment of immune cells in the mammal. The immune cells may be any immune cells, including but not limited to, one or more of lymphocytes and granulocytes. Preferably, the immune cells include one or more of neutrophils, T cells (e.g., CD4⁺, CD8⁺, and γδ T cells), natural killer (NK) cells, and B cells. The inventive methods of reducing the activation or recruitment of immune cells in the mammal may, advantageously, reduce lung remodeling, reduce inflammation, treat or prevent a viral disease, and/or promote the survival of a mammal infected with a virus and/or suffering from a viral disease.

Suppressing the activation of immune cells includes reducing the maturation, proliferation, and/or the migration of immune cells, e.g., to a specific locale (e.g., the site of an antigen or the site of chemotactic cytokine production, such as CCL2, CCL3, CCL5, CCL19, CCL20, CCL21, etc.). Suppressing the activation of immune cells can be measured by the lack of production of cytokines associated with the activation of immune cells. In particular, the IL-21 blocking agent may suppress the immune cell production of cytokines such as, for example, any or all of interleukin (IL)-6, IL-8, IL-12, (e.g., IL-12p70), IL-1 (e.g., IL-1β), IL-10, IL-17 (e.g., IL-17α), IL-18, IL-23, tumor necrosis factors (TNF) (e.g., TNFα), and/or chemokines (e.g., CXCL10, CXCL8, CXCL1, CCL1, CCL2, CCL3, CCL5, CCL7, CCL8, CCL13, CCL17, CCL18, CCL20, and/or CCL22). Assays for measuring or detecting a decrease in the activation and/or recruitment of immune cells are known in the art.

Another embodiment of the invention provides a method of decreasing the expression of at least one cytokine and/or chemokine in a mammal comprising administering to the mammal an IL-21 blocking agent in an amount effective to decrease the expression of the cytokine and/or chemokine. The cytokine and/or chemokine may be any of the cytokines or chemokines described herein. A preferred embodiment of the invention provides a method of decreasing the expression of at least one cytokine and/or chemokine in a mammal comprising administering to the mammal an IL-21 blocking agent in an amount effective to decrease the expression of the cytokine and/or chemokine, wherein the at least one cytokine and/or chemokine is selected from the group consisting of interferon (IFN)-γ, IL-6, CXCL1, IL-17α, and Without being bound to a particular theory, it is believed that IL-6, IL-17α, IFN-γ, and IL-1β are associated with Th17 and Tc17 responses after PVM infection and that CXCL1 promotes neutrophil recruitment during PVM infection. The inventive methods of decreasing the expression of any one or more of (IFN)-γ, IL-6, CXCL1, IL-17α, and IL-1βmay, advantageously, reduce the activation or recruitment of immune cells, reduce lung remodeling, reduce inflammation, treat or prevent a viral disease, and/or promote the survival of a mammal infected with a virus and/or suffering from a viral disease.

Still another embodiment of the invention provides a method of decreasing the expression of at least one protein in a mammal comprising administering to the mammal an IL-21 blocking agent in an amount effective to decrease the expression of the protein, wherein the at least one protein is selected from the group consisting of matrix metalloproteinase-8 (MMP8) and S100 calcium binding protein A8 (S100A8). MMP8 and S100A8 are produced by neutrophils. Without being bound to a particular theory, it is believed that MMP8 and S100A8 are involved in mediating the inflammatory response and/or lung remodeling. The inventive methods of decreasing the expression of MMP8 and/or S100A8 may, advantageously, reduce the activation or recruitment of immune cells, reduce lung remodeling, reduce inflammation, treat or prevent a viral disease, and/or promote the survival of a mammal infected with a virus and/or suffering from a viral disease.

The IL-21 blocking agent can be any agent that inhibits the biological activity of IL-21. The biological activity of IL-21 may be inhibited in any manner, e.g., by inhibiting the expression of any one or more of IL-21 mRNA, IL-21 protein, IL-21R mRNA, and IL-21R protein; by inhibiting the binding of IL-21 to IL-21R, and/or by inhibiting IL-21 signaling, as compared to that which is observed in the absence of the IL-21 blocking agent. The biological activity may be inhibited to any degree that realizes a beneficial therapeutic effect. For example, in some embodiments, the biological activity may be completely inhibited (i.e., prevented), while in other embodiments, the biological activity may be partially inhibited (i.e., reduced). As used herein, unless stated otherwise, the terms “IL-21” and “IL-21R” refer to IL-21 and IL-21R, respectively, in any form (e.g., mRNA or protein) and from any species (e.g., human or mouse).

In an embodiment of the invention, the IL-21 blocking agent is an agent that inhibits IL-21 signaling. IL-21 signaling can be inhibited in any manner. For example, the IL-21 blocking agent may inhibit the activation or activity of any one or more of various downstream targets of IL-21 signaling (e.g., proteins in the JAK-STAT pathway (e.g., any one or more of the JAK kinases (e.g., JAK1 and JAK3), STAT proteins (e.g., STAT1, STAT3, STAT5A, and STAT5B), and the proteins in the phosphoinositol 3-kinase (PI 3-kinase) and MAP kinase pathways). For example, the IL-21 blocking agent may be an agent that binds to the IL-21 protein, thereby reducing or preventing IL-21 signaling and inhibiting its function. By way of illustration, the agent that inhibits IL-21 signaling can be any of the antibodies or antibody fragments, antisense nucleic acids, or chemical inhibitors (e.g., small molecule or peptide (or polypeptide) inhibitor) described herein.

In an embodiment, the IL-21 blocking agent is an agent that inhibits the binding of IL-21 to the IL-21 receptor (IL-21R). In this regard, the IL-21 blocking agent may be an agent that binds to the IL-21 protein or the IL-21R protein, thereby reducing or preventing the binding of the IL-21 protein to the IL-21R and inhibiting its function, as well as agents that compete with the IL-21 protein for the native IL-21 binding site of the IL-21 receptor. By way of illustration, the agent that inhibits the binding of IL-21 to the IL-21 receptor can be any of the antibodies or antibody fragments, antisense nucleic acids, or chemical inhibitors (e.g., small molecule or peptide inhibitor) described herein.

In an embodiment of the invention, the IL-21 blocking agent is an antibody or antibody fragment that specifically binds to IL-21 or IL-21R. Anti-IL-21 and anti-IL-21R antibodies and antibody fragments can be monoclonal or polyclonal. Anti-IL-21 and anti-IL-21R antibodies and antibody fragments can be prepared using the IL-21 and IL-21R proteins disclosed herein and routine techniques. Examples of such antibodies or antibody fragments include those specific to the native IL-21 binding site of the IL-21 receptor or a functional domain of IL-21 (e.g., the IL-21R binding portion of IL-21).

Chemical inhibitors of IL-21 include small molecules and peptides or polypeptides that inhibit IL-21 signaling, bind the IL-21 or IL-21R protein or functional fragment thereof, or compete with the IL-21 protein or functional fragment thereof for its native binding site of the IL-21R. Suitable inhibitors can include, for example, chemical compounds or a non-active fragment or mutant of an IL-21 protein. In this regard, in an embodiment of the invention, the IL-21 blocking agent is a mutated IL-21. The mutation may include any insertions, deletions, and/or substitutions of one or more amino acids in any position of the IL-21 protein that effectively inhibits IL-21 biological activity (e.g., IL-21 signaling and/or binding of IL-21 to IL-21R). For example, the chemical inhibitor can bind to the IL-21R and/or inhibit IL-21 signaling. In this regard, the IL-21 blocking agent may be a chemical inhibitor. In a preferred embodiment, the IL-21 blocking agent inhibits the activation or activity of any one or more of a JAK kinase, a STAT protein, a phosphoinositol 3-kinase (PI 3-kinase) and a MAP kinase, as described herein.

Chemical inhibitors of IL-21 can be identified using routine techniques. For example, chemical inhibitors can be tested in binding assays to identify molecules and peptides (or polypeptides) that bind to IL-21 or IL-21R with sufficient affinity to inhibit IL-21 biological activity (e.g., binding of IL-21 to IL-21R, and/or IL-21 signaling). Also, competition assays can be performed to identify small-molecules and peptides (or polypeptides) that inhibit the activation of downstream targets of IL-21 signaling or compete with IL-21 or functional fragment thereof for binding to its native binding site of IL-21R. Such techniques could be used in conjunction with mutagenesis of the IL-21 protein or functional fragment thereof itself, and/or with high-throughput screens of known chemical inhibitors.

The functional fragment of the IL-21 or IL-21R protein can comprise any contiguous part of the IL-21 or IL-21R protein that retains a relevant biological activity of the IL-21 or IL-21R protein, e.g., binds to IL-21R or IL-21 and/or participates in IL-21 signaling. Any given fragment of an IL-21 or IL-21R protein can be tested for such biological activity using methods known in the art. For example, the functional fragment can comprise, consist essentially of, or consist of the IL-21R binding portion of the IL-21 protein or the IL-21 binding portion of the IL-21R protein. In reference to the parent IL-21 or IL-21R protein, the functional fragment preferably comprises, for instance, about 10% or more, 25% or more, 30% or more, 50% or more, 60% or more, 80% or more, 90% or more, or even 95% or more of the parent IL-21 protein.

In an embodiment of the invention, the IL-21 blocking agent is any suitable agent that inhibits the expression of any one or more of IL-21 mRNA, IL-21 protein, IL-21R mRNA, and IL-21R protein. The IL-21 blocking agent can be a nucleic acid at least about 10 nucleotides in length that specifically binds to and is complementary to a target nucleic acid encoding any one or more of IL-21 mRNA, IL-21 protein, IL-21R mRNA, and IL-21R protein or a complement thereof. The IL-21 blocking agent may be introduced into a host cell, wherein the cell is capable of expressing any one or more of IL-21 mRNA, IL-21 protein, IL-21R mRNA, and IL-21R protein, in an effective amount for a time and under conditions sufficient to interfere with expression of any one or more of IL-21 mRNA, IL-21 protein, IL-21R mRNA, and IL-21R protein, respectively. In some embodiments, RNA interference (RNAi) is employed. In this regard, the IL-21 blocking agent may comprise an RNAi agent. In an embodiment, the RNAi agent may comprise a small interfering RNA (siRNA), a short hairpin miRNA (shMIR), a microRNA (miRNA), or an antisense nucleic acid. The RNAi agent, e.g., siRNA, shRNA, miRNA, and/or antisense nucleic acid can comprise overhangs. That is, not all nucleotides need bind to the target sequence. RNA interference nucleic acids employed can be at least about 19, at least about 40, at least about 60, at least about 80, at least about 100, at least about 120, at least about 140, at least about 160, at least about 180, at least about 200, at least about 220, at least about 240, from about 19 to about 250, from about 40 to about 240, from about 60 to about 220, from about 80 to about 200, from about 60 to about 180, from about 80 to about 160, and/or from about 100 to about 140 nucleotides in length.

The RNAi agent, e.g., siRNA or shRNA, can be encoded by a nucleotide sequence included in a cassette, e.g., a larger nucleic acid construct such as an appropriate vector. Examples of such vectors include lentiviral and adenoviral vectors, as well as other vectors described herein with respect to other aspects of the invention. An example of a suitable vector is described in Aagaard et al. Mol. Ther., 15(5): 938-45 (2007). When present as part of a larger nucleic acid construct, the resulting nucleic acid can be longer than the comprised RNAi nucleic acid, e.g., greater than about 70 nucleotides in length. In some embodiments, the RNAi agent employed cleaves the target mRNA. In other embodiments, the RNAi agent employed does not cleave the target mRNA.

Any type of suitable siRNA, miRNA, and/or antisense nucleic acid can be employed. In an embodiment, the antisense nucleic acid comprises a nucleotide sequence complementary to at least about 8, at least about 15, at least about 19, or from about 19 to about 22 nucleotides of a nucleic acid encoding any one or more of IL-21 mRNA, IL-21 protein, IL-21R mRNA, and IL-21R protein or a complement thereof. In an embodiment, the siRNA may comprise, e.g., trans-acting siRNAs (tasiRNAs) and/or repeat-associated siRNAs (rasiRNAs). In another embodiment, the miRNA may comprise, e.g., a short hairpin miRNA (shMIR).

In an embodiment of the invention, the IL-21 blocking agent may inhibit or downregulate to some degree the expression of the protein encoded by an IL-21R or IL-21 gene, e.g., at the DNA, RNA, or other level of regulation. In this regard, a host cell comprising an IL-21 blocking agent expresses none of any one or more of IL-21 mRNA, IL-21 protein, IL-21R mRNA, and IL-21R protein or lower levels of any one or more of IL-21 mRNA, IL-21 protein, IL-21R mRNA, and IL-21R protein as compared to a host cell that lacks an IL-21 blocking agent. In accordance with an embodiment of the invention, the IL-21 blocking agent, such as an RNAi agent, such as a shMIR, can target a nucleotide sequence of an IL-21 or IL-21R gene or mRNA encoded by the same.

In an embodiment, the IL-21 sequence is a human IL-21 sequence. For example, human IL-21 is assigned Gene NCBI Entrez Gene ID No. 59067, and an Online Mendelian Inheritance in Man (OMIM) No. 605384. The human IL-21 gene is found on chromosome 4 at 4q26-q27. Two transcriptional variants include mRNA GenBank Accession Nos: NM_{—}021803.2 (SEQ ID NO: 1) and NM_{—}001207006.1 (SEQ ID NO: 2), with corresponding protein sequences GenBank Accession Nos: NP_{—}068575.1 (SEQ ID NO: 3) and NP_{—}001193935.1 (SEQ ID NO: 4), respectively. Human genomic IL-21 sequences include GenBank Accession Nos: NC_{—}000004.11, AC_{—}000136.1, AC053545.5, AY763518.1, CH471056.2, CS080568.1, CS237090.1, CS450761.1, CS582814.1, FB677173.1, GM619752.1, HB976773.1, HC196689.1, HC203173.1, HC203434.1, HC686913.1, and JA104585.1. Human IL-21 mRNA sequences also include Genbank Accession Nos: AF254069.1, BC066258.1, BC066259.1, BC066260.1, BC066261.1, BC066262.1, BC069124.1, CD559460.2, and DQ645417.1. Human IL-21 amino acid sequences include Genbank Accession Nos: AAU88182.1, EAX05226.1, CAI94500.1, CAJ47524.1, CAL81203.1, CAN87399.1, CAS03522.1, CAV33288.1, CBE74752.1, CBI70418.1, CBI85469.1, CBI85472.1, CBL93962.1, CCA63962.1, AAG29348.1, AAH66258.1, AAH66259.1, AAH66260.1, AAH66261.1, AAH66262.1, AAH69124.1, and ABG36529.1. Other human sequences, as well as other IL-21 species can be employed in accordance with the invention.

In an embodiment, the IL-21R sequence is a human IL-21R sequence. For example, human IL-21R is assigned Gene NCBI Entrez Gene ID No. 50615, and an Online Mendelian Inheritance in Man (OMIM) No. 605383. The human IL-21R gene is found on chromosome 16 at 16p11. Three transcriptional variants include mRNA GenBank Accession Nos: NM_{—}021798.3 (SEQ ID NO: 7), NM_{—}181078.2 (SEQ ID NO: 8), and NM_{—}181079.4 (SEQ ID NO: 9), with corresponding protein sequences GenBank Accession Nos: NP_{—}068570.1 (SEQ ID NO: 10), NP_{—}851564.1 (SEQ ID NO: 11), and NP_{—}851565.4 (SEQ ID NO: 12), respectively. Human genomic IL-21R sequences include GenBank Accession Nos: NC_{—}000016.9, AC_{—}000148.1, AC002303.1, AC004525.1, AY064474.1, CH471145.2, CS080576.1, CS450755.1, FB702445.1, HB976766.1, HC005802.1, HC196703.1, HC202963.1, HC203224.1, HC686703.1, HI574132.1, HI574134.1, and HI574136.1. Human IL-21R mRNA sequences also include Genbank Accession Nos: AA354979.1, AF254067.1, AF269133.1, AK292663.1, AK312825.1, and AW576566.1. Human IL-21R amino acid sequences include Genbank Accession Nos: AAL39168.1, EAW55746.1, EAW55747.1, EAW55748.1, EAW55749.1, CAI94502.1, CAL81201.1, CAS03334.1, CBE74748.1, CBG76750.1, CBI70421.1, CBI85467.1, CBI85470.1, CBL93960.1, CBX47555.1, CBX47556.1, CBX47557.1, AAG29346.1, AAG23419.1, BAF85352.1, and BAG35682.1. Other human sequences, as well as other IL-21R species can be employed in accordance with the invention.

In another embodiment, the IL-21 sequence is a mouse sequence. For example, mouse IL-21 is assigned Gene NCBI Entrez Gene ID No. 60505. The mouse IL-21 gene is found on chromosome 3 at 3B. A transcript includes mRNA Genbank Accession No.: NM 021782.2 (SEQ ID NO: 5), with corresponding protein sequence NP_{—}068554.1 (SEQ ID NO: 6). Mouse genomic IL-21 sequences include Genbank Accession Nos: NT_{—}039248.1, NC_{—}000069.5, NT 039252.1, AC_{—}000025.1, AL645807.4, AL645966.30, AL645982.26, AL662823.12, and CH466530.1. Mouse IL-21 mRNA sequences also include Genbank Accession Nos: AF254070.1, AY428162.1, BC125414.1, BC125416.1, and DQ645418.1. Mouse IL-21 amino acid sequences include Genbank Accession Nos: CAM28076.1, CAI26234.1, CAM18421.1, EDL35100.1, AAG29349.1, AAR06254.1, AAI25415.1, AAI25417.1, and ABG36530.1. Other mouse sequences, as well as other IL-21 species can be employed in accordance with the invention.

In an embodiment, the IL-21R sequence is a mouse sequence. For example, mouse IL-21R is assigned Gene NCBI Entrez Gene ID No. 60504. The mouse IL-21R gene is found on chromosome 7 at 7 F4. A transcript includes mRNA Genbank Accession No.: NM_{—}021887.2 (SEQ ID NO: 13), with corresponding protein sequence NP_{—}068687.1 (SEQ ID NO: 14). Mouse genomic IL-21R sequences include Genbank Accession Nos: NC_{—}000073.5, AC_{—}000029.1, AC125213.3, CH466531.1, CS450758.1, FB702451.1, HB976768.1, HC005885.1, HC202965.1, HC203226.1, and HC686705.1. Mouse IL-21R mRNA sequences also include Genbank Accession Nos: AB049137.1, AF254068.1, AF269134.1, AF279436.1, AF477982.1, AF477983.1, AF477984.1, AF477985.1, AF477986.1, AK040073.1, AK137793.1, AK150824.1, AK171826.1, and AK172032.1. Mouse IL-21R amino acid sequences include Genbank Accession Nos: EDL17325.1, EDL17326.1, EDL17327.1, CAL81202.1, CAS03337.1, CBE74750.1, CBG76754.1, CBI85468.1, CBI85471.1, CBL93961.1, BAB13736.1, AAG29347.1, AAG23420.1, AAF86350.1, AAL82632.1, AAL82633.1, AAL82634.1, AAL82635.1, AAL82636.1, BAE29886.1, BAE42685.1, and BAE42787.1. Other mouse sequences, as well as other IL-21R species can be employed in accordance with the invention. Human and mouse antisense nucleic acids are commercially available (e.g., from OriGene Technologies, Inc., Rockville, Md. or Sigma-Aldrich, St. Louis, Mo.) and can be prepared using the nucleic acid sequences encoding the IL-21 or IL-21R proteins disclosed herein and routine techniques.

In accordance with an embodiment of the invention, the IL-21 blocking agent, such as an RNAi agent, such as a shMIR, can target a nucleotide sequence selected from the group consisting of the 5′ untranslated region (5′ UTR), the 3′ untranslated region (3′ UTR), and the coding sequence of IL-21 or IL-21R, complements thereof, and any combination thereof. Any suitable IL-21 or IL-21R target sequence can be employed. In an embodiment of the invention, the sequences of the IL-21 blocking agent can be designed against a human IL-21 with Accession No. NM_{—}001207006.1 (SEQ ID NO: 2) but also recognize NM_{—}021803.2 (SEQ ID NO: 1) (or vice-versa). In an embodiment of the invention, the sequences of the IL-21 blocking agent can be designed against human IL-21R with any one of Accession Nos: NM_{—}021798.3 (SEQ ID NO: 7), NM_{—}181078.2 (SEQ ID NO: 8), and NM_{—}181079.4 (SEQ ID NO: 9), but also recognize either of the other two sequences. In still another embodiment, the sequences of the IL-21 blocking agent can be designed against a mouse IL-21 with Accession No. NM_{—}021782.2 (SEQ ID NO: 5) or a mouse IL-21R with Accession No. NM_{—}021887.2 (SEQ ID NO: 13). RNAi agents can be designed against any appropriate IL-21 or IL-21R mRNA sequence.

In another embodiment, the IL-21 blocking agent is an IL-21 receptor/Fc fusion protein. The IL-21 receptor/Fc fusion protein is a soluble variation of the native IL-21R which binds IL-21 protein, thereby competing with the native, cell surface IL-21R for binding to IL-21. Accordingly, the IL-21 receptor/Fc fusion protein may inhibit the binding of IL-21 to the native IL-21R. The IL-21 receptor/Fc fusion protein may also inhibit the activation or activity of any one or more of various downstream targets of IL-21 signaling (e.g., proteins in the JAK-STAT pathway (e.g., any one or more of the JAK kinases (e.g., JAK1 and JAK3), STAT proteins (e.g., STAT1, STAT3, STAT5A, and STAT5B), and proteins in the phosphoinositol 3-kinase (PI 3-kinase) and MAP kinase pathways). The IL-21 receptor/Fc fusion protein may be from any mammal. In a preferred embodiment, the IL-21 receptor/Fc fusion protein is a mouse IL-21 receptor/Fc fusion protein or a human IL-21 receptor/Fc fusion protein. A suitable human IL-21 receptor/Fc fusion protein is recombinant human IL-21R subunit Fc chimera, available from R&D Systems, Minneapolis, Minn.

The IL-21 blocking agent can be obtained by methods known in the art. For example, IL-21 blocking agents that are peptides or polypeptides can be obtained by de novo synthesis as described in references, such as Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westw000d et al., Oxford University Press, Oxford, United Kingdom, 2000; and U.S. Pat. No. 5,449,752. Also, IL-21 blocking agents can be recombinantly produced using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Further, the IL-21 blocking agent can be isolated and/or purified from a natural source, e.g., a human. Methods of isolation and purification are well-known in the art. In this respect, the IL-21 blocking agents may be exogenous and can be synthetic, recombinant, or of natural origin.

The IL-21 blocking agents that are peptides or polypeptides can be glycosylated, amidated, carboxylated, phosphorylated, esterified, N-acylated, cyclized via, e.g., a disulfide bridge, or converted into an acid addition salt and/or optionally dimerized or polymerized, or conjugated. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Of course, the method of the invention can comprise administering two or more IL-21 blocking agents, any of which may be the same or different from one another. Furthermore, the IL-21 blocking agent can be provided as part of a larger polypeptide construct. For instance, the IL-21 blocking agent can be provided as a fusion protein comprising an IL-21 blocking agent along with other amino acid sequences or a nucleic acid encoding same. The IL-21 blocking agent also can be provided as part of a conjugate or nucleic acid encoding same. Conjugates, as well as methods of synthesizing conjugates in general, are known in the art (See, for instance, Hudecz, F., Methods Mol. Biol. 298: 209-223 (2005) and Kirin et al., Inorg. Chem. 44(15): 5405-5415 (2005)).

The IL-21 blocking agent can be administered to the mammal by administering a nucleic acid encoding the IL-21 blocking agent to the mammal. “Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

Nucleic acids encoding the IL-21 blocking agent (and degenerate nucleic acid sequences encoding the same amino acid sequences), can be constructed based on chemical synthesis and/or enzymatic ligation reactions using procedures known in the art. See, for example, Sambrook et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides).

The nucleic acids can be incorporated into a recombinant expression vector. For purposes herein, the term “recombinant expression vector” means a genetically-modified oligonucleotide or polynucleotide construct that permits the expression of an mRNA or polypeptide by a host cell, when the construct comprises a nucleotide sequence encoding the mRNA or polypeptide, and the vector is contacted with the cell under conditions sufficient to have the mRNA or polypeptide expressed within the cell. The vectors are not naturally-occurring as a whole. However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages does not hinder the transcription or replication of the vector.

The recombinant expression vector can be any suitable recombinant expression vector, and can be used to transform or transfect any suitable host. Suitable vectors include those designed for propagation and expansion or for expression or both, such as plasmids and viruses. The vector can be of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI01, pBI101.2, pBI101.3, pBIl21 and pBIN19 (Clontech). Examples of animal expression vectors include pEUK-Cl, pMAM and pMAMneo (Clontech). Preferably, the recombinant expression vector is a viral vector, e.g., a retroviral vector.

The recombinant expression vectors can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papilloma virus, and the like.

Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or normative promoter and/or stop codon operably linked to the nucleotide sequence encoding the IL-21 blocking agent, or to the nucleotide sequence which is complementary to the nucleotide sequence encoding the IL-21 blocking agent. The selection of stop codons and promoters, e.g., strong, weak, inducible, tissue-specific and developmental-specific, is within the ordinary skill of the artisan. Similarly, the combining of a nucleotide sequence with a stop codon and a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The IL-21 blocking agent and nucleic acids encoding them can be of synthetic or natural origin, and can be isolated or purified to any degree. The terms “isolated” and “purified” as used herein means having been increased in purity, wherein “purity” is a relative term, and not to be necessarily construed as absolute purity. For example, the purity can be at least about 50%, can be greater than 60%, 70% or 80%, or can be 100%.

The methods described herein may be used for any purpose, e.g., the treatment or prevention of disease, especially viral pneumonia. The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of a viral disease in a mammal. Furthermore, the treatment or prevention provided by the inventive method can include treatment or prevention of one or more conditions or symptoms of the viral disease, e.g., pneumonia, being treated or prevented. Also, for purposes herein, “prevention” can encompass delaying the onset of the disease, or a symptom or condition thereof. With respect to the inventive methods, the pneumonia can be any pneumonia, including any of the pneumonias caused by any of the viruses discussed herein.

For purposes of the invention, the amount or dose of the IL-21 blocking agent administered should be sufficient to effect the desired biological response, e.g., a therapeutic or prophylactic response, in the mammal over a reasonable time frame. The dose will be determined by the efficacy of the particular IL-21 blocking agent and the condition of the mammal (e.g., human), as well as the body weight of the mammal (e.g., human) to be treated. The dose of the IL-21 blocking agent also will be determined by the existence, nature and extent of any adverse side effects that might accompany the administration of a particular IL-21 blocking agent. Typically, the attending physician will decide the dosage of the IL-21 blocking agent with which to treat each individual patient, taking into consideration a variety of factors, such as age, body weight, general health, diet, sex, IL-21 blocking agent to be administered, route of administration, and the severity of the condition being treated.

The mammal referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human. The mammal can be non-diseased, a mammal afflicted with a disease, such as pneumonia, or a mammal predisposed to a disease, such as pneumonia.

Administering an IL-21 blocking agent to the mammal in accordance with the inventive methods may comprise administering a pharmaceutical composition comprising the IL-21 blocking agent and a pharmaceutically acceptable carrier. The carrier can be any of those conventionally used and is limited only by chemico-physical considerations, such as solubility and lack of reactivity with the active compound(s), and by the route of administration. The pharmaceutically acceptable carriers described herein, for example, vehicles, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use. The choice of carrier will be determined in part by the particular compounds used in the pharmaceutical composition, as well as by the particular method used to administer the IL-21 blocking agent.

In an embodiment of the invention, administering the IL-21 blocking agent to the mammal may comprise administering the IL-21 blocking agent orally, intravenously, intramuscularly, subcutaneously, or intraperitoneally. The following formulations for oral, intravenous, intramuscular, subcutaneous, or intraperitoneal administration are exemplary and are in no way limiting. More than one route can be used to administer the IL-21 blocking agent, and in certain instances, a particular route can provide a more immediate and more effective response than another route.

Oral formulations may include any suitable carrier. For example, formulations suitable for oral administration may comprise suitable carriers, such as lactose, sucrose, starch, talc magnesium stearate, crystalline cellulose, methyl cellulose, carboxymethyl cellulose, glycerin, sodium alginate or gum arabic among others.

Intravenous, intramuscular, subcutaneous, or intraperitoneal formulations may include any suitable carrier. For example, formulations suitable for intravenous, intramuscular, subcutaneous, or intraperitoneal administration may comprise sterile aqueous solutions of the IL-21 blocking agent with solutions which are preferably isotonic with the blood of the recipient. Such formulations may be prepared by dissolving the IL-21 blocking agent in water containing physiologically compatible substances such as sodium chloride (e.g. 0.1-2.0M), glycine, and the like, and having a buffered pH compatible with physiological conditions to produce an aqueous solution, and rendering said solution sterile.

The following examples further illustrate the invention but, of course, should not be construed as in any way limiting its scope.

### EXAMPLES

### Mice

Wild type C57B1/6 mice were obtained from the Jackson Laboratory (Bar Harbor, Me.). Il21r^{-/-} mice (knockout mice that do not express the Il-21r gene) (Ozaki et al., Science, 298:1630-1634 (2002)) were analyzed at 8-12 weeks of age. Mice expressing the Il2-EmGFP/Il21-mCherry recombineered Bac reporter transgene have been described (Wang et al., PNAS, 108:9542-9547 (2011)). Transgenic mice expressing the human IL-21 cDNA under the control of the H-2K^{b} promoter and IgM enhancer have been described (Ozaki et al., J. Immunol., 173:5361-5371 (2004)). All experiments were performed under protocols approved by the NHLBI and NIAID Animal Care and Use Committees, and followed NIH guidelines for use of animals in intramural research.

Virus Inoculation.

Virus stocks (PVM strain J3666) were prepared as previously described (Domachowske et al., J. Immunol., 165:2677-2682 (2000)). Mice were anesthetized briefly via inhalation of 20% halothane and were inoculated intranasally with 12-60 plaque-forming units (PFU) of Pneumonia Virus of Mice (PVM) in 50-100 μl of PBS. IL-21R/Fc fusion protein was obtained from R&D Systems (Minneapolis, Minn.). Human Fc Cγl control protein was obtained from BioXCell (Kuala Lumpur, Malaysia).

Histology.

Lungs were inflated before excision, fixed in 10% formalin, embedded in paraffin, 5 μm thick sections were cut and slides were stained with hematoxylin and eosin. Clinical scores for morphology and inflammation and edema were evaluated.

Bronchoalveolar Lavage Fluid and Lung Cell Preparation.

Lungs were inflated intratracheally with 1 ml cold 0.1% bovine serum albumin (BSA) in phosphate buffered saline (abbreviated PBS) and recovered fluid was used for enzyme-linked immunosorbent assay (ELISA), and to prepare BAL cells. Lung tissue was minced into small pieces using a razor blade and digested in a solution containing 0.5 mg/ml Liberase (Roche, Basel, Switzerland) and 0.5 mg/ml DNase I (Sigma-Aldrich, St. Louis, Mo.) in serum free Roswell Park Memorial Institute medium (RPMI) for 30 min at 37° C. Digested tissue was then pushed through a cell strainer with a syringe. Cells were centrifuged, and red blood cells (RBCS) were lysed with ACK, followed by two washes with complete RPMI.

Flow Cytometric Analysis.

Single cell suspensions from BAL fluid or lung tissue were surface stained in FACS (fluorescence-activated cell sorting) buffer (PBS containing 0.5% BSA and 0.02% azide) using antibodies from BD Biosciences (Franklin Lakes, N.J.). For intracellular staining of cytokines, cells were activated with PMA (10 ng/ml) and ionomycin (1 μM) (Sigma-Aldrich) for 4 h in the presence of BD GOLGIPLUG protein transport inhibitor (BD Biosciences). Cells were first surface-stained with anti-CD8, fixed, and permeabilized with BD CYTOFIX/CYTOPERM solution (BD Biosciences). Data were acquired with either a FACSCANTO II flow cytometer or an BD LSR II flow cytometer and were analyzed with FLOWJO software.

Dendritic Cell Isolation.

Splenic dendritic cells were isolated by collagenase digestion followed by positive selection with pan-DC microbeads (Miltenyi Biotec, Bergisch Gladbach, Germany). Splenic DCs were seeded at 2×10⁶/sample in 24-well plates, rested at 37° C. for 1 h, and stimulated with cytokines. Supernatants were collected, and IL-6 levels were measured by ELISA (BD Biosciences).

RNA Preparation and Real Time PCR.

RNA was extracted from lung tissue by homogenization in TRIZOL solution (Invitrogen, Leek, The Netherlands) followed by RNA cleanup with the RNeasy kit (Qiagen, Hilden, Germany). RNA was reverse-transcribed using Omniscript (Qiagen). Polymerase chain reaction (PCR) reactions to quantitatively detect cytokine and chemokine RNAs used probe sets from Applied Biosystems (Carlsbad, Calif.) and the 7900HT Sequence Detection System. Relative levels of PVM SH gene expression (Percopo et al., J. Immunol., 183:604-612 (2009)) were determined by quantitative reverse transcriptase (RT)-PCR, with normalization to Rpl7 expression.

ELISAs.

Lung tissue was homogenized in cold PBS and tissue debris was removed by centrifugation (14,000 rpm for 10 min). Levels of CXCL1 in equal volumes of BAL fluid and equal amounts of protein from lung homogenates were measured using a kit (Antigenix America, Huntington Station, N.Y.).

Statistical analysis. Two-tailed paired t-tests were performed using Prism 4.0 (GRAPHPAD software).

### Example 1

This example demonstrates that IL-21 expression is induced following infection with PVM.

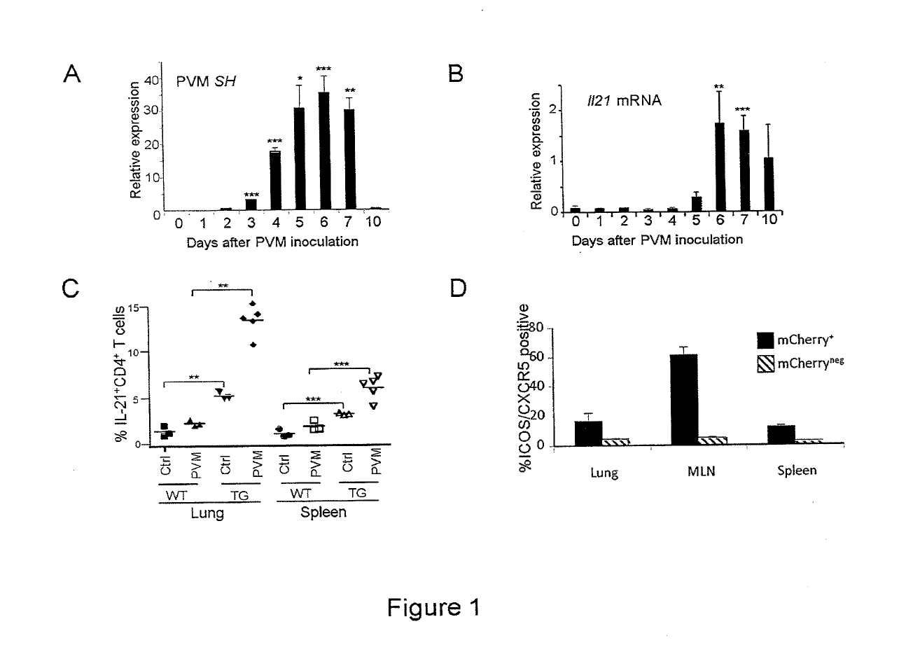

To determine whether IL-21 is expressed in the lung in response to PVM infection, wild type (WT) mice were inoculated intra-nasally with PVM virions, and Il-21 mRNA expression was measured in the lung at time points during the 10 days following infection. Expression of the PVM SH gene as an indicator of virus burden was greatest at day 6 after inoculation (FIG. 1A). As expected, infected mice developed pneumonia and began to succumb to infection starting at day 7 and SH gene expression declined, with it no longer being detected among survivors at day 10 (FIG. 1A). Il-21 mRNA was detected as early as day 5, with a peak at day 6, and levels declined thereafter (FIG. 1B). Interestingly, at day 10, virus was no longer detected in lung tissue, but Il-21 mRNA could be detected in the surviving mice.

To identify IL-21-producing cells, transgenic reporter mice were used in which the mCherry reporter is inserted at the translation start site of the Il-21 gene in a bacterial artificial chromosome (BAC) clone by recombineering technology. These mice were inoculated with PVM, and single cell suspensions from lung tissue and lymphoid organs were examined ex vivo by flow cytometry. mCherry expression was detected in approximately 5% of CD4⁺ T cells in lung tissue of uninfected mice, with expression increasing to approximately 10-15% at day 6 after inoculation (FIG. 1C), which corresponded to peak Il-21 mRNA expression (FIG. 1B), whereas mCherry expression was not detected in NK1.1⁺ cells (NK cells or NKT cells), γδ T cells, or CD8⁺ T cells in the lung. In addition to this acute response in the lung, a systemic immune response also developed, as indicated by the increase in mCherry expression from approximately 1.9% of splenic CD4⁺ T cells in uninfected reporter mice (TG Ctrl) to approximately 5.9% at day 6 (TG PVM) (FIG. 1C). CD4⁺ T cells expressing IL-21 were enriched for expression of the ICOS/CXCR5 surface markers characteristic of T follicular helper (Tfh) cells (FIG. 1D) compared to CD4⁺ T cells that did not express IL-21, with a particularly high percentage of Tfh cells in mediastinal lymph node IL-21 expressors.

### Example 2

This example demonstrates reduced lung inflammation in response to PVM infection in Il21r^{-/-} mice.

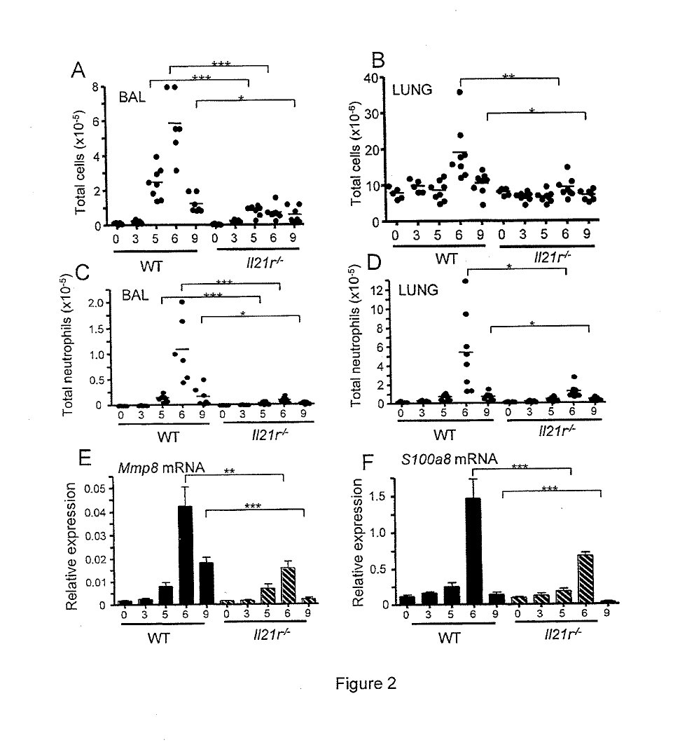

The increased numbers of IL-21-producing CD4⁺ T cells in the lung after PVM infection suggested that this cytokine might participate in antiviral host defense and/or viral pathogenesis. To investigate this possibility, WT and Il21r^{-/-} mice were infected with PVM and cellular inflammatory responses were evaluated. Microscopy revealed that lung tissue from PVM-infected WT mice exhibited progressive, severe inflammation (severe cell infiltration) diffusely through the lung at days 5 and 6, whereas lungs from Il21r^{-/-} mice had only mild and focal perivascular inflammation at these time points. Microscopy also revealed that infiltration of both neutrophils and lymphocytes were evident in the WT mice. Bronchoalveolar lavage (BAL) fluid from PVM-infected mice was also examined and significantly fewer cells were found in the BAL fluid from the Il21r^{-/-} mice than from WT mice on days 5, 6 and 9 post-inoculation (FIG. 2A), as well as fewer cells in lung parenchyma at days 6 and 9 (FIG. 2B). Granulocytic infiltration is a hallmark of the early inflammatory response to PVM, and there were fewer Ly6G⁺CD11b⁺neutrophils in the BAL fluid at days 5, 6 and 9 (FIG. 2C) and lung parenchyma (FIG. 2D) at day 6 in the Il21r^{-/-} than WT mice inoculated with PVM. Consistent with this, at day 6 post-inoculation, Il21r^{-/-} mice also had significantly decreased levels of mRNAs encoding matrix metalloprotease-8 (MMP8) and S100A8 (FIGS. 2E and 2F), two neutrophil-derived proteins involved in mediating the inflammatory/lung remodeling response (Passey et al., J. Leukoc. Biol., 66:549-556 (1999); Greenlee et al., Physiol. Rev., 87:69-98 (2007)).

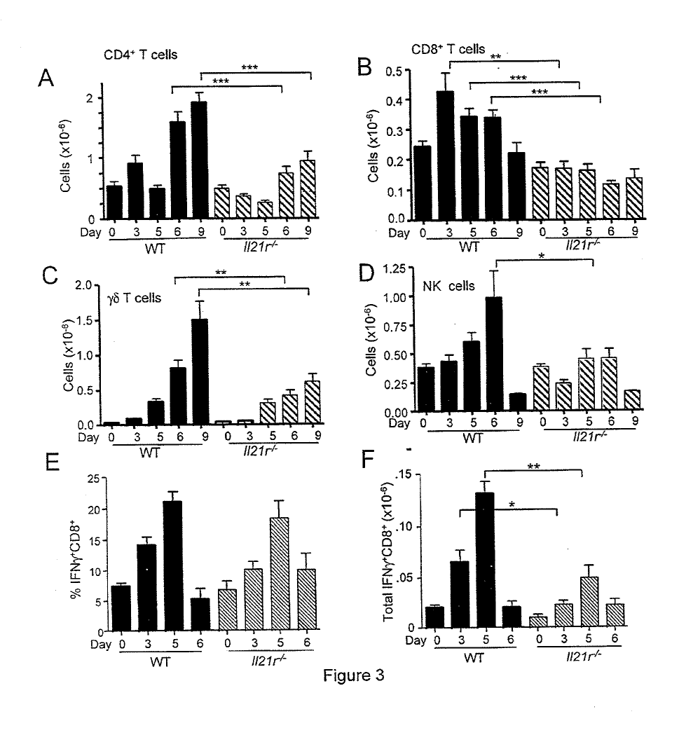

Because T cells were reported to be important for the resolution of sub-lethal PVM infection (Frey et al., J. Virol., 82:11619-11627 (2008)), the presence of CD4⁺ T cells, CD8⁺ T cells, γδ T cells, and NK cells was assessed in the lung during the course of PVM infection in WT and Il21r^{-/-} mice (FIG. 3). CD4⁺ T cell infiltration was apparent by day 6 after inoculation, but to a greater degree in WT mice than in Il21r^{-/-} mice, with elevated CD4⁺ T cell numbers persisting through day 9 post-infection (FIG. 3A). Increased numbers of CD8⁺ T cells were observed in the lungs of WT mice as early as 3 days after PVM inoculation but then declined, whereas fewer CD8⁺ T cells were observed in the lungs of infected Il21r^{-/-} mice (FIG. 3B). Although γδ T cells infiltrated both WT and Il21r^{-/-} lungs equivalently starting on day 5, fewer γδ⁺ T cells were seen in the Il21r^{-/-} lungs at days 6 and 9 (FIG. 3C). NK cell numbers were also significantly lower in the lung parenchyma in the Il21r^{-/-} mice (FIG. 3D). Thus, the recruitment of CD4⁺, CD8⁺, and γδ T cells, as well as NK cells was diminished in the absence of IL-21 signaling.

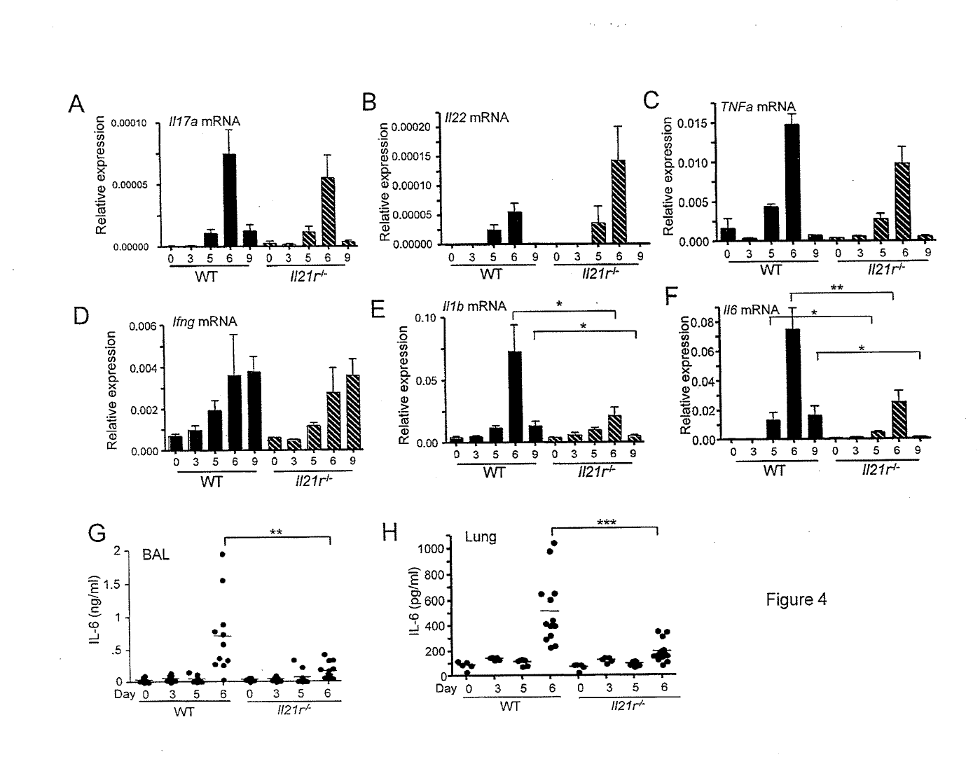

CD8⁺ T cell function was previously reported to be suppressed in PVM-infected lungs (Claassen et al., J. Immunol., 175:6597-6604 (2005)). Interestingly, the percentage of lung CD8⁺ T cells producing IFNγ increased during the course of infection in both WT and Il21r^{-/-} mice (FIG. 4E), but because of the reduced number of CD8⁺ T cells in the lungs of Il21r^{-/-} mice (FIG. 4B), the total number of IFNγ-producing CD8⁺ T cells was significantly diminished (FIG. 4F). PVM peptides that allow the detection of viral-specific CD8⁺ T cells have not been identified; thus, these CD8⁺ T cells may include both PVM-specific cytotoxic cells as well as bystander cells activated by the inflammatory environment.

### Example 3

This example demonstrates reduced levels of IL-6 in lungs of PVM-infected Il21r−/− mice.

The inflammatory response to PVM in WT mice includes rapid infiltration of both neutrophils and lymphocytes into the lung, and, as noted above, these responses were reduced in Il21r^{-/-} mice. Neutrophil infiltration in PVM infection is coordinated by multiple cytokines and proinflammatory chemokines (Bonville et al., J. Virol., 77:1237-1244 (2003); Bonville et al., J. Virol., 78:7984-7989 (2004)). In other settings, IL-17 has been shown to promote neutrophil responses, leading to the induction of cytokines and chemokines that augment the levels of neutrophil progenitors and the subsequent expansion of peripheral neutrophil populations (Ye et al., J. Exp. Med., 194:519-527 (2001)). Because IL-21 promotes the differentiation of IL-17-producing cells (Korn et al., Nature, 448:484-487 (2007); Nurieva et al., Nature 448:480-483 (2007); Zhou et al., Nat. Immunol., 8:967-974 (2007)), the levels of cytokines associated with Th17 and Tc17 responses after PVM infection were measured. Levels of both Il17a (FIG. 4A) and Il22 (FIG. 4B) mRNAs in lung tissue were not significantly different in WT and Il21r^{-/-} PVM-infected mice. Tnfa (FIG. 4C) and Ifng (FIG. 4D) mRNAs tended to be slightly lower in the Il21r^{-/-} mice at day 6 after inoculation, but the differences were not statistically significant. I11b mRNA was significantly decreased at days 6 and 9 (FIG. 4E), although differences in IL-1β protein were not observed. However, Il6 mRNA was significantly decreased at days 5, 6, and 9 (FIG. 4F), with a corresponding decrease in IL-6 protein at day 6 in both BAL fluid and lung tissue (FIGS. 4G and 4H). These results suggest that IL-21 signaling may directly or indirectly control the production of IL-6 during PVM infection.

### Example 4

This example demonstrates diminished levels of CXCL1 in PVM infected Il21r^{-/-} mice.

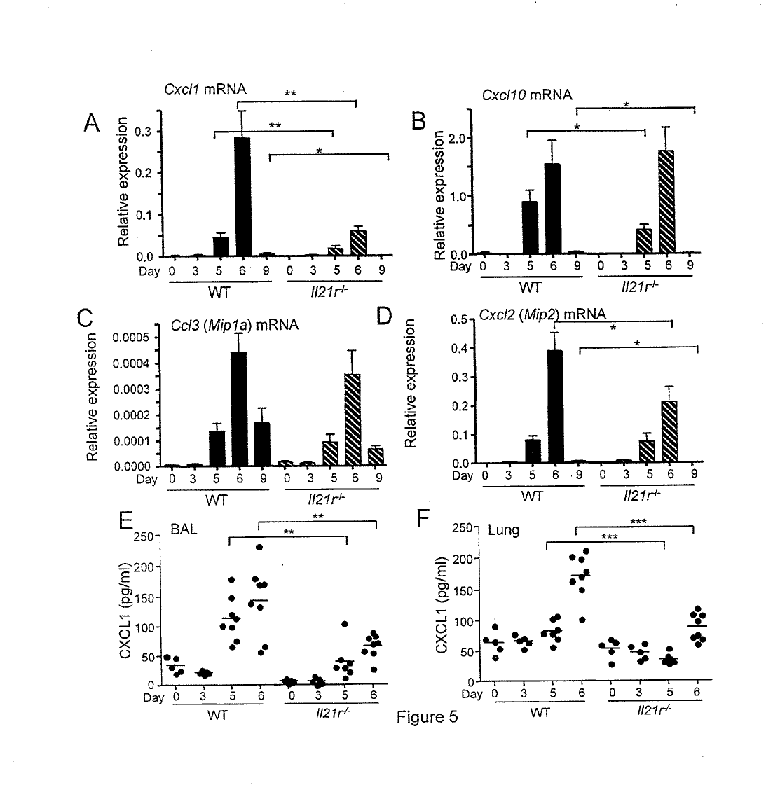

Because the accumulation of neutrophils in the lungs was diminished in PVM-infected Il21r^{-/-} mice (FIG. 2), the expression of several chemokines known to promote neutrophil recruitment during PVM infection (Gabryszewski et al., J. Immunol., 186:1151-Il61 (2011)) was examined. Interestingly, Cxcl1 mRNA was less potently induced in lung tissue of Il21r^{-/-} mice than in WT mice during PVM infection (FIG. 5A), whereas mRNA encoding CXCL10, a chemokine involved in lymphocyte recruitment, was induced with slightly more rapid kinetics in WT mice (FIG. 5B), but by day 6 the levels were not significantly different in WT vs the Il21r^{-/-} mice. No significant difference in the expression of the chemokine CCL3 (also known as macrophage inflammatory protein-1α, MIP-1α), which has been implicated in neutrophil recruitment and immunomodulatory protection in PVM infection, was observed (FIG. 5C), and although lower Cxcl2 mRNA expression was observed in Il21r^{-/-} mice at day 6 (FIG. 5D), there was no significant difference in CXCL2 protein expression in the lung. Corresponding to the diminished levels of Cxcl1 mRNA (FIG. 5A), CXCL1 protein levels were significantly lower in BAL fluid (FIG. 5E) and lung homogenate (FIG. 5F) at days 5 and 6 after PVM infection of the Il21r^{-/-} mice, correlating with the reduced neutrophil infiltration in the lungs of these mice.

### Example 5

This example demonstrates similar immune responses in lung draining lymph nodes from PVM-infected Il21r^{-/-} mice.

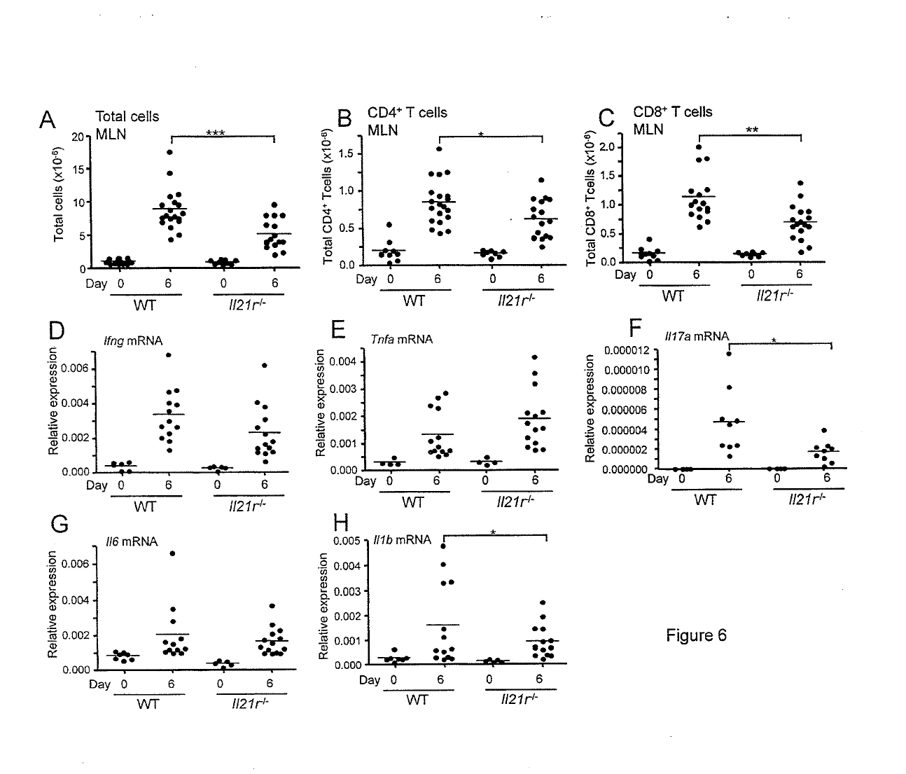

Because of the roles that IL-21 plays in the development of cellular immune responses, it was of interest to determine whether changes in cellularity, chemokines, and cytokines were specific to the lung response or whether they reflected an overall deficiency of the immune response in Il21r^{-/-} mice. Lymphoid populations were thus examined in the mediastinal lymph nodes (MLN) at days 0 and 6 after PVM infection and it was found that both WT and Il21r^{-/-} MLN undergo T cell expansion (FIG. 6A), including both CD4⁺ (FIG. 6B) and CD8⁺ (FIG. 6C) in response to infection, although this was reduced in the Il21r^{-/-} MLN (FIGS. 6A, 6B, and 6C). Similar levels of Ifng, Tnfa, and Il6 mRNAs were measured in the MLN, although Il17a and Il1b mRNA levels were lower in the Il21r⁻¹⁻ MLN (FIG. 6D-6H). These data indicate that although lymphoid expansion was lower in the Il21r⁻¹⁻ MLN, most inflammatory cytokine responses were similar, suggesting that the cellular immune response was not completely defective in the knockout (KO) mice.

### Example 6

This example demonstrates that constitutive expression of IL-21 leads to increased cellular infiltration to the lung and increased IL-6 production.

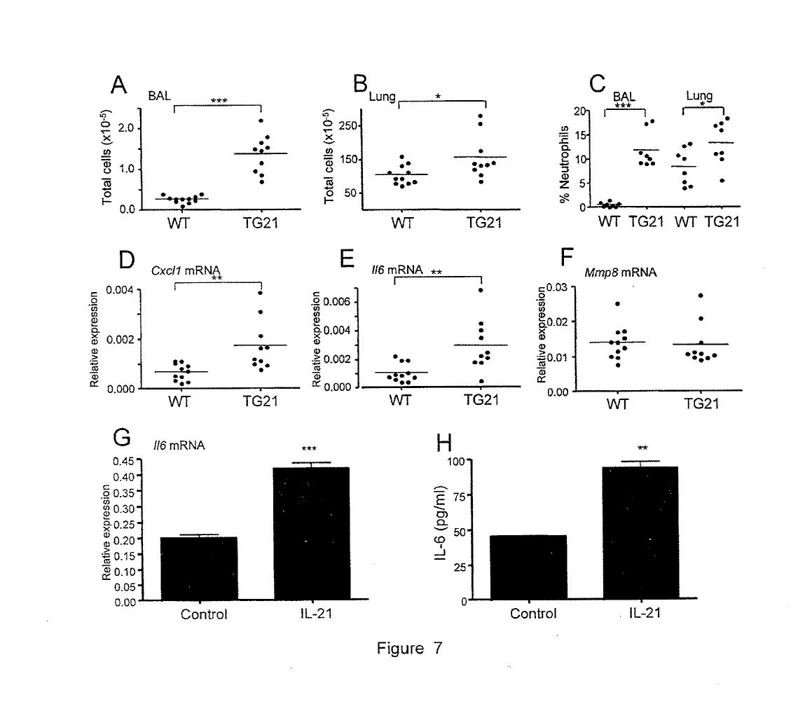

The production of IL-21 by CD4⁺ T cells in the normal lung suggested that IL-21 may play a role in normal lung homeostasis as well as in the development of the inflammatory response to PVM infection. To investigate this possibility, lung cellularity was examined in transgenic mice that constitutively express IL-21 in immune cells (TG21). Increased cellularity was observed in both the BAL fluid (FIG. 7A) and the lung (FIG. 7B), with an elevated percentage of neutrophils in both BAL and lung in the uninfected TG21 mice (FIG. 7C). Levels of Cxcl1 mRNA (FIG. 7D) and Il6 mRNA (FIG. 7E) were both increased in the TG21 lungs. Interestingly, however, levels of the neutrophil-expressed Mmp8 mRNA were similar in WT and TG21 lungs (FIG. 7F), suggesting that the enhanced recruitment of neutrophils to the lung occurred without an associated inflammatory response.

In order to determine whether IL-21 could directly induce the production of IL-6, dendritic cell populations were purified and it was found that IL-21 significantly induced both Il-6/IL-6 mRNA and protein (FIGS. 7G and 7H) in these cells.

### Example 7

This example demonstrates prolonged survival of PVM-infected Il21r^{-/-} mice.

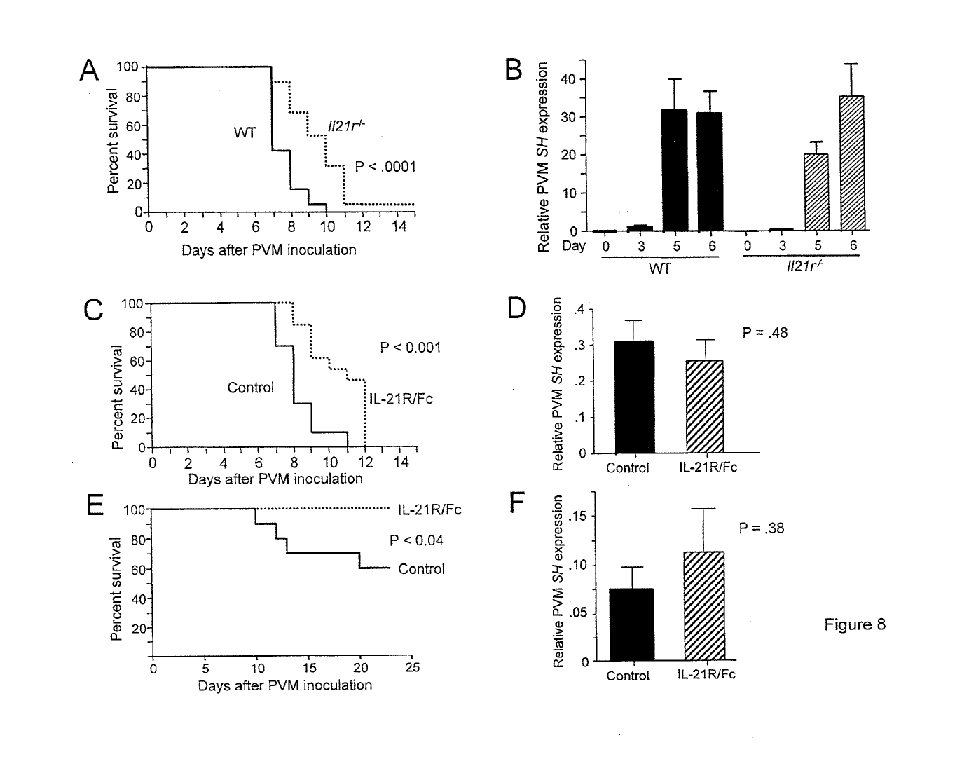

Above, an increase in IL-21 production in response to PVM infection as well as decreased neutrophil and lymphocyte accumulation and diminished production of IL-6 in Il21r^{-/-} mice was observed. It was unclear, however, whether IL-21 mediated the PVM pathogenic response or instead promoted host-defense. To clarify the role of IL-21, WT and Il21r⁻⁻ mice were inoculated intra-nasally with a dose of PVM previously shown to be sufficient to kill WT mice, and determined their survival (FIG. 8A). As anticipated, 60% of the WT mice died on day 7 and all were dead by day 10 after PVM inoculation. In contrast, 90% of the Il21r^{-/-} mice were still alive at day 7, and 30% of the Il21r^{-/-} mice survived beyond day 10, with 5% (1 of 19 mice) surviving through day 21. Thus, the absence of IL-21 signaling conferred a survival advantage (p<0.0001), even though there was no significant difference in PVM SH gene expression, as an indicator of virus copy number, in WT versus Il21r^{-/-} lungs (FIG. 8B). These results suggest that IL-21 does not have a significant effect on viral replication or clearance but that it promotes the inflammatory response following infection with PVM, with earlier and augmented mortality in response to PVM infection in WT mice.

### Example 8

This example demonstrates prolonged survival of PVM-infected WT mice pre-treated with IL-21R/Fc fusion protein.

To determine whether the enhanced survival of the Il21r^{-/-} mice was a direct result of the effects of IL-21 in the lung during the response to PVM infection, an IL-21R/Fc fusion protein was used to block IL-21 activity. When WT mice were intratracheally treated with the IL-21R/Fc fusion protein one day prior and 2 days after PVM inoculation, there was significantly higher survival than in mice treated with an Fc control protein (FIG. 8C), even though no significant differences in virus replication, based on SH gene expression, were detected between these two groups at day 6 of infection (FIG. 8D). Moreover, when mice were inoculated with a lower dose of PVM, treatment with the IL-21R/Fc fusion protein conferred complete protection (FIG. 8E), and again there were no differences in viral burden (FIG. 8F). These data indicate that the survival advantage seen in Il21r⁻¹⁻ mice did not result from developmental differences but rather resulted from the lack of IL-21 signaling.

### Example 9

This example demonstrates prolonged survival of WT mice treated with IL-21R/Fc fusion protein after PVM infection.

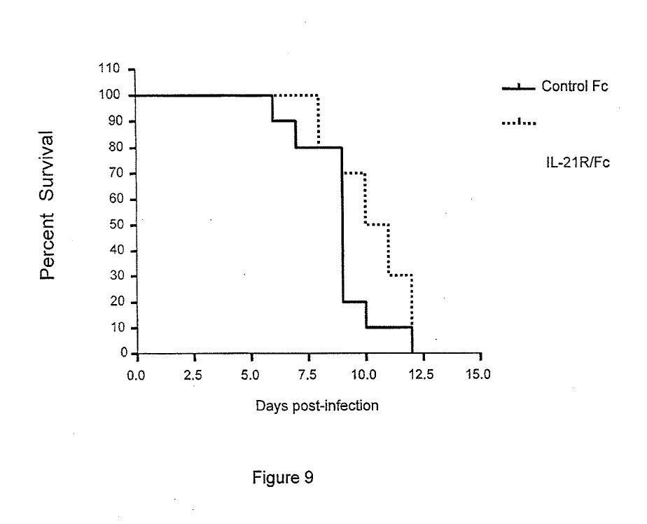

Wildtype B6 mice were infected intranasally with PVM at time 0. Mice received 50 μg of either IL-21R/Fc or control Fc intratracheally on days 3 and 4 post-infection and their survival was monitored. When WT mice were treated with the IL-21R/Fc fusion protein one days 3 and 4 after PVM inoculation, there was higher survival than in mice treated with an Fc control protein (FIG. 9).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing a viral disease in a mammal comprising administering to the mammal an interleukin (IL)-21 blocking agent in an amount effective to treat or prevent the viral disease in the mammal.

2. A method of prolonging the survival of a mammal suffering from a viral disease comprising administering to the mammal an interleukin (IL)-21 blocking agent in an amount effective to prolong the survival of the mammal suffering from the viral disease.

3. The method of claim 1, wherein the viral disease is caused by a virus selected from the group consisting of herpes viruses, pox viruses, hepadnaviruses, papilloma viruses, adenoviruses, coronoviruses, orthomyxoviruses, paramyxoviruses, flaviviruses, and caliciviruses.

4. The method of claim 1, wherein the viral disease is caused by a virus selected from the group consisting of pneumonia virus of mice (PVM), respiratory syncytial virus (RSV), influenza virus, herpes simplex virus, Epstein-Barr virus, varicella virus, cytomegalovirus, hepatitis A virus, hepatitis B virus, hepatitis C virus, human T-lymphotropic virus, calicivirus, adenovirus, and Arena virus.

5. The method of claim 1, wherein the viral disease is selected from the group consisting of influenza, pneumonia, herpes, hepatitis, hepatitis A, hepatitis B, hepatitis C, chronic fatigue syndrome, sudden acute respiratory syndrome (SARS), gastroenteritis, enteritis, carditis, encephalitis, bronchiolitis, respiratory papillomatosis, meningitis, and mononucleosis.

6. The method of claim 1, wherein the viral disease is a pulmonary viral disease.

7. The method of claim 6, wherein the pulmonary viral disease is pneumonia.

8. The method of claim 7, wherein the pneumonia is caused by at least one virus selected from the group consisting of pneumonia virus of mice (PVM), respiratory syncytial virus (RSV), influenza, herpes, and varicella.

9. The method of claim 8, wherein the pneumonia is caused by PVM or RSV.

10. A method of reducing the activation or recruitment of immune cells in a mammal comprising administering to the mammal an IL-21 blocking agent in an amount effective to reduce the activation or recruitment of immune cells in the mammal.

11. A method of decreasing the expression of at least one cytokine and/or chemokine in a mammal comprising administering to the mammal an IL-21 blocking agent in an amount effective to decrease the expression of the cytokine and/or chemokine, wherein the at least one cytokine and/or chemokine is selected from the group consisting of interferon (IFN)-γ, IL-6, CXCL1, IL-17α, and IL-1β.

12. A method of decreasing the expression of at least one protein in a mammal comprising administering to the mammal an IL-21 blocking agent in an amount effective to decrease the expression of the protein, wherein the at least one protein is selected from the group consisting of MMP8 and S100A8.

13. The method of claim 1, wherein the IL-21 blocking agent is an agent that inhibits the binding of IL-21 to the IL-21 receptor (IL-21R).

14. The method of claim 1, wherein the IL-21 blocking agent is an agent that inhibits IL-21 signaling.

15. The method of claim 1, wherein the IL-21 blocking agent is an agent that inhibits the expression any one or more of IL-21 mRNA, IL-21 protein, IL-21R mRNA, and IL-21R protein.

16. The method of claim 15, wherein the IL-21 blocking agent is an RNA interference (RNAi) agent.

17. The method of claim 1, wherein the IL-21 blocking agent is an IL-21 receptor/Fc fusion protein.

18. The method of claim 1, wherein the IL-21 blocking agent is an antibody or antibody fragment that specifically binds to IL-21 or IL-21R.

19. The method of claim 1, wherein the IL-21 blocking agent is a mutated IL-21.

20. The method of claim 1, wherein the IL-21 blocking agent is a chemical inhibitor.

21. The method of claim 14, wherein the IL-21 blocking agent inhibits the activation or activity of any one or more of a JAK kinase, a STAT protein, a phosphoinositol 3-kinase (PI 3-kinase) and a MAP kinase.

22. The method of claim 1, wherein the mammal is a mouse.

23. The method of claim 1, wherein the mammal is a human.

24-46. (canceled)


SEQUENCE LISTING (14 sequences)

SEQ ID NO: 1 - DNA, 616 bp, Homo sapiens
ctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacactatgttacatactctaatatagtagtgaaagtcatttctttgtattccaagtggaggag

SEQ ID NO: 2 - DNA, 706 bp, Homo sapiens
ctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaaggtatctaccttaagtttcatttgattttctgctttatctttacctatccagatttgcttcttagttactcacggtatactatttccacagatgattcatcagcatctgtcctctagaacacacggaagtgaagattcctgaggatctaacttgcagttggacactatgttacatactctaatatagtagtgaaagtcatttctttgtattccaagtggaggag

SEQ ID NO: 3 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 4 - protein, 153 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 5 - DNA, 3072 bp, Mus musculus
gagaaccagaccaaggccctgtcatcagctcctggagactcagttctggtggcatggagaggacccttgtctgtctggtagtcatcttcttggggacagtggcccataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaagatgattcatcagcatctctcctagaacacataggacccgaagattcctgaggatccgagaagattcccgaggactgaggagacgccggacactatagacgctcacgaatgcaggagtacatcttgcctcttgggattgcaagtggagaagtacgatacgttatgataagaacaactcagaaaagctataggttaagatcctttcgcccattaactaagcagacattgtggttccctgcacagactccatgctgtcaacatggaaaatctcaactcaacaagagcccagcttcccgtgtcagggatttctggtgcttctcaagctgtggcttcatcttattgcccaactgtgacattctttgattggaaggggaaaactaaagcttttagcaaaaatacagctagggaatttgtcgatctgcgagagtaagacctcttatgatcctaacggaatgatgtaagctggaaataataagcataagatgaaattgaaaattgaagtctttattctttaagaaaaactttgtacttgaaagcatgtctgaagagtttactcattaccacaaacatctagcatattgataactaacatctttatactctacaagagaggctttccagataggtacagtttttcttctctattaggtctatcaaaatttaacctattatgagggtcacccctggctttcactgtttttctaaagaggcaagggtgtagtaagaagcaggcttaagttgccttcctcccaatgtcaagttcctttataagctaatagtttaatcttgtgaagatggcaatgaaagcctgtggaagtgcaaacctcactatcttctggagccaagtagaattttcaagtttgtagctctcacctcaagtggttatgggtgtcctgtgatgaatctgctagctccagcctcagtctcctctcccacatcctttcctttctttcctctttgaaacttctaagaaaaagcaatccaaacaagttcagcacttaagacacattgcatgcacacttttgataagttaaatccaaccatctatttaaaatcaaaatcaggagatgagccaagagaccagaggttctgttccagttttaaacagacttttactgaacatcccaatcttttaaccacagaggctaaattgagcaaatagttttgccatttgatataatttccaacagtatgtttcaatgtcaagttaaaaagtctacaaagctattttccctggagtggtatcatcgctttgagaatttcttatggttaaaatggatctgagatccaagcatggcctgggggatggttttgatctaaggaaaaaggtgtctgtacctcacagtgcctttaaaacaagcagagatcccgtgtaccgccctaagatagcacagactagtgttaactgattcccagaaaagtgtcacaatcagaaccaacgcattctcttaaactttaaaaatatgtattgcaaagaacttgtgtaactgtaaatgtgtgactgttgatgacattatacacacatagcccacgtaagtgtccaatggtgctagcattggttgctgagtttgctgctcgaaagctgaagcagagatgcagtccttcacaaagcaatgatggacagagaggggagtctccatgttttattcttttgttgtttctggctgtgtaactgttgacttcttgacattgtgatttttatatttaagacaatgtatttattttggtgtgtttattgttctagccttttaaatcactgacaatttctaatcaagaagtacaaataattcaatgcagcacaggctaagagcttgtatcgtttggaaaagccagtgaaggcttctccactagccatgggaaagctacgctttagagtaaactagacaaaattgcacagcagtcttgaacctctctgtgctcaagactcagccagtcctttgacattattgttcactgtgggtgggaacacattggacctgacacactgttgtgtgtccatgaaggttgccactggtgtaagctttttttggttttcattctcttatctgtagaacaagaatgtggggctttcctaagtctattctgtattttattctgaacttcgtatgtctgagttttaatgttttgagtactcttacaggaacacctgaccacacttttgagttaaattttatcccaagtgtgatatttagttgttcaaaaagggaagggatatacatacatacatacatacatacatacatatatatatatatatatacatatatatatatatatatatgtatatatatatatatatagagagagagagagagagagagagagaaagagagagaggttgttgtaggtcataggagttcagaggaaatcagttatggccgttaatactgtagctgaaagtgttttctttgtgaataaattcatagcattattgatctatgttattgctctgttttatttacagtcacacctgagaatttagttttaatatgaatgatgtactttataacttaatgattatttattatgtatttggttttgaatgtttgtgttcatggcttcttatttaagacctgatcatattaaatgctacccagtcagaa

SEQ ID NO: 6 - protein, 146 aa, Mus musculus [sequence omitted]

SEQ ID NO: 7 - DNA, 4512 bp, Homo sapiens
ctagaggcagctcagatagaggagctctttgggaagagacgccgggcaggcagaagcagcaggtaccccctccacatccctagggctctgtgatgtaggcagaggcccgtgggagtcagcatgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatgaggctgactggatgtccagagctggccaggccactgggccctgagccagagacaaggtcacctgggctgtgatgtgaagacacctgcagcctttggtctcctggatgggcctttgagcctgatgtttacagtgtctgtgtgtgtgtgtgcatatgtgtgtgtgtgcatatgcatgtgtgtgtgtgtgtgtgtcttaggtgcgcagtggcatgtccacgtgtgtgtgtgattgcacgtgcctgtgggcctgggataatgcccatggtactccatgcattcacctgccctgtgcatgtctggactcacggagctcacccatgtgcacaagtgtgcacagtaaacgtgtttgtggtcaacagatgacaacagccgtcctccctcctagggtcttgtgttgcaagttggtccacagcatctccggggctttgtgggatcagggcattgcctgtgactgaggcggagcccagccctccagcgtctgcctccaggagctgcaagaagtccatattgttccttatcacctgccaacaggaagcgaaaggggatggagtgagcccatggtgacctcgggaatggcaattttttgggcggcccctggacgaaggtctgaatcccgactctgataccttctggctgtgctacctgagccaagtcgcctcccctctctgggctagagtttccttatccagacagtggggaaggcatgacacacctgggggaaattggcgatgtcacccgtgtacggtacgcagcccagagcagaccctcaataaacgtcagcttccttccttctgcggccagagccgaggcgggcgggggtgagaacatcaatcgtcagcgacagcctgggcacccgcggggccgtcccgcctgcagagggccactcgggggggtttccaggcttaaaatcagtccgtttcgtctcttggaaacagctccccaccaaccaagatttctttttctaacttctgctactaagtttttaaaaattccctttatgcacccaagagatatttattaaacaccaattacgtagcaggccatggctcatgggacccaccccccgtggcactcatggagggggctgcaggttggaactatgcagtgtgctccggccacacatcctgctgggccccctaccctgccccaattcaatcctgccaataaatcctgtcttatttgttcatcctggagaattgaagggaggtcaagttgtttgtcaatgatttgtcagagaacctgttgaaatgtgaattaagaagctaagaaaatatttcttagcaacattttctttttcttttttttttttttcttttgagacagagtctcactctcgtcgcccaggctggaatgcagtggtgcgatctcggctctctgcaacctctgtctcccgggttcaagcgatttcctgcgtcagccccagagtagctggaattacaggcacacaccaccacgcctggctaatttttgtatttttagtagagctggggccaccctggcccggccccgtcttcctccccaaaggtcagactgcaggctgcagggctgtgctggaggagccagctctagctcacccatgcttttgcaacagggtcgggttggaagtcagcacaggtcagtcctgcggaaggttccttcgtgactcatctgtgaagtggggtggttgggagaggtagctgagagaatgcatgagagtcctcggtgcctggcaggaggctggaaggttctagaacactgatggttataagagtgggactgtgagcctgggatcggggggtgtgagacttggatgggagcacaagagtggaaacacagcttctgcacggagcaggcgcagccctcaacaccccgtgcacctgcaccctagggactcttgggtccagatgtgctgtggttttcacaccttcttgggggcaacaggttccaggagccacctgtgggtgccacctgagccacaggctcccaggaaagcagcacagctctcctgcacccagagcttgctgggtggcggaggggaacacagatggttggggaaggcctgaggccagattgggggactctggactggggcagatgaggctcctcagaatcccacctttgaagggaactcagcttataaacacagaggagcaaagttggagggccgggcgtagtggctcacacctgtgatctcagcactttgggaggccaaggaaggtggatcacttgaggccaggagttcgagaccagcctgggcaacatagcaaggccccatctctacaaaaattattattttttaaaaaaattagccaggtgtggtggtgcttgcctatagtcccagctactcgggaggctaaggtgggaggatcgctggagcccaggaatttgaggctgcagtgagctgtgattacaccgttgcactccagcctgggtcacagatcaagaccctgtctcttaaaaataaaagttggagacaagagctggctcacctgaaaggagggattagtaggtaggagggtggatggaggatggatggatgtgtgggtggataggaagatggtattaagttggtgcaaaagtctttgatattactcttaatggctttaataaaaagcttgaaggaagaatgattggttggatagacagagataaatgcatactggaaacaaagataaagataaaacacaagttataccaggccagcaactctattttgttcactgcctttagtcccagcctggcacatagtaggcactcaataaagcctgatttgtagcaaaaaaaaaaaaaaaaa

SEQ ID NO: 8 - DNA, 4865 bp, Homo sapiens
ctcttcctccccactctgcacatgcggctgggtggcagccagcggcctcagacagacccactggcgtctctctgctgagtgaccgtaagctcggcgtctggccctctgcctgcctctccctgagtgtggctgacagccacgcagctgtgtctgtctgtctgcggcccgtgcatccctgctgcggccgcctggtaccttccttgccgtctctttcctctgtctgctgctctgtgggacacctgcctggaggcccagctgcccgtcatcagagtgacaggtcttatgacagcctgattggtgactcgggctgggtgtggattctcaccccaggcctctgcctgctttctcagaccctcatctgtcacccccacgctgaacccagctgccacccccagaagcccatcagactgcccccagcacacggaatggatttctgagaaagaagccgaaacagaaggcccgtgggagtcagcatgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatgaggctgactggatgtccagagctggccaggccactgggccctgagccagagacaaggtcacctgggctgtgatgtgaagacacctgcagcctttggtctcctggatgggcctttgagcctgatgtttacagtgtctgtgtgtgtgtgtgcatatgtgtgtgtgtgcatatgcatgtgtgtgtgtgtgtgtgtcttaggtgcgcagtggcatgtccacgtgtgtgtgtgattgcacgtgcctgtgggcctgggataatgcccatggtactccatgcattcacctgccctgtgcatgtctggactcacggagctcacccatgtgcacaagtgtgcacagtaaacgtgtttgtggtcaacagatgacaacagccgtcctccctcctagggtcttgtgttgcaagttggtccacagcatctccggggctttgtgggatcagggcattgcctgtgactgaggcggagcccagccctccagcgtctgcctccaggagctgcaagaagtccatattgttccttatcacctgccaacaggaagcgaaaggggatggagtgagcccatggtgacctcgggaatggcaattttttgggcggcccctggacgaaggtctgaatcccgactctgataccttctggctgtgctacctgagccaagtcgcctcccctctctgggctagagtttccttatccagacagtggggaaggcatgacacacctgggggaaattggcgatgtcacccgtgtacggtacgcagcccagagcagaccctcaataaacgtcagcttccttccttctgcggccagagccgaggcgggcgggggtgagaacatcaatcgtcagcgacagcctgggcacccgcggggccgtcccgcctgcagagggccactcgggggggtttccaggcttaaaatcagtccgtttcgtctcttggaaacagctccccaccaaccaagatttctttttctaacttctgctactaagtttttaaaaattccctttatgcacccaagagatatttattaaacaccaattacgtagcaggccatggctcatgggacccaccccccgtggcactcatggagggggctgcaggttggaactatgcagtgtgctccggccacacatcctgctgggccccctaccctgccccaattcaatcctgccaataaatcctgtcttatttgttcatcctggagaattgaagggaggtcaagttgtttgtcaatgatttgtcagagaacctgttgaaatgtgaattaagaagctaagaaaatatttcttagcaacattttctttttcttttttttttttttcttttgagacagagtctcactctcgtcgcccaggctggaatgcagtggtgcgatctcggctctctgcaacctctgtctcccgggttcaagcgatttcctgcgtcagccccagagtagctggaattacaggcacacaccaccacgcctggctaatttttgtatttttagtagagctggggccaccctggcccggccccgtcttcctccccaaaggtcagactgcaggctgcagggctgtgctggaggagccagctctagctcacccatgcttttgcaacagggtcgggttggaagtcagcacaggtcagtcctgcggaaggttccttcgtgactcatctgtgaagtggggtggttgggagaggtagctgagagaatgcatgagagtcctcggtgcctggcaggaggctggaaggttctagaacactgatggttataagagtgggactgtgagcctgggatcggggggtgtgagacttggatgggagcacaagagtggaaacacagcttctgcacggagcaggcgcagccctcaacaccccgtgcacctgcaccctagggactcttgggtccagatgtgctgtggttttcacaccttcttgggggcaacaggttccaggagccacctgtgggtgccacctgagccacaggctcccaggaaagcagcacagctctcctgcacccagagcttgctgggtggcggaggggaacacagatggttggggaaggcctgaggccagattgggggactctggactggggcagatgaggctcctcagaatcccacctttgaagggaactcagcttataaacacagaggagcaaagttggagggccgggcgtagtggctcacacctgtgatctcagcactttgggaggccaaggaaggtggatcacttgaggccaggagttcgagaccagcctgggcaacatagcaaggccccatctctacaaaaattattattttttaaaaaaattagccaggtgtggtggtgcttgcctatagtcccagctactcgggaggctaaggtgggaggatcgctggagcccaggaatttgaggctgcagtgagctgtgattacaccgttgcactccagcctgggtcacagatcaagaccctgtctcttaaaaataaaagttggagacaagagctggctcacctgaaaggagggattagtaggtaggagggtggatggaggatggatggatgtgtgggtggataggaagatggtattaagttggtgcaaaagtctttgatattactcttaatggctttaataaaaagcttgaaggaagaatgattggttggatagacagagataaatgcatactggaaacaaagataaagataaaacacaagttataccaggccagcaactctattttgttcactgcctttagtcccagcctggcacatagtaggcactcaataaagcctgatttgtagcaaaaaaaaaaaaaaaaa

SEQ ID NO: 9 - DNA, 5006 bp, Homo sapiens
ctcttcctccccactctgcacatgcggctgggtggcagccagcggcctcagacagacccactggcgtctctctgctgagtgaccgtaagctcggcgtctggccctctgcctgcctctccctgagtgtggctgacagccacgcagctgtgtctgtctgtctgcggcccgtgcatccctgctgcggccgcctggtaccttccttgccgtctctttcctctgtctgctgctctgtgggacacctgcctggaggcccagctgcccgtcatcagagtgacaggtcttatgacagcctgattggtgactcgggctgggtgtggattctcaccccaggcctctgcctgctttctcagaccctcatctgtcacccccacgctgaacccagctgccacccccagaagcccatcagactgcccccagcacacggaatggatttctgagaaagaagccgaaacagaagatgaggcaatgaggctgcgagaggtagagtgattttccctcggtgactcaactgggacgtagcaggtcgggcagtcaagccaggtgaccccatgagctgtcgctgcatctttctcatgaagcacggggaacgggtcggatggcccgtgggagtcagcatgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatgaggctgactggatgtccagagctggccaggccactgggccctgagccagagacaaggtcacctgggctgtgatgtgaagacacctgcagcctttggtctcctggatgggcctttgagcctgatgtttacagtgtctgtgtgtgtgtgtgcatatgtgtgtgtgtgcatatgcatgtgtgtgtgtgtgtgtgtcttaggtgcgcagtggcatgtccacgtgtgtgtgtgattgcacgtgcctgtgggcctgggataatgcccatggtactccatgcattcacctgccctgtgcatgtctggactcacggagctcacccatgtgcacaagtgtgcacagtaaacgtgtttgtggtcaacagatgacaacagccgtcctccctcctagggtcttgtgttgcaagttggtccacagcatctccggggctttgtgggatcagggcattgcctgtgactgaggcggagcccagccctccagcgtctgcctccaggagctgcaagaagtccatattgttccttatcacctgccaacaggaagcgaaaggggatggagtgagcccatggtgacctcgggaatggcaattttttgggcggcccctggacgaaggtctgaatcccgactctgataccttctggctgtgctacctgagccaagtcgcctcccctctctgggctagagtttccttatccagacagtggggaaggcatgacacacctgggggaaattggcgatgtcacccgtgtacggtacgcagcccagagcagaccctcaataaacgtcagcttccttccttctgcggccagagccgaggcgggcgggggtgagaacatcaatcgtcagcgacagcctgggcacccgcggggccgtcccgcctgcagagggccactcgggggggtttccaggcttaaaatcagtccgtttcgtctcttggaaacagctccccaccaaccaagatttctttttctaacttctgctactaagtttttaaaaattccctttatgcacccaagagatatttattaaacaccaattacgtagcaggccatggctcatgggacccaccccccgtggcactcatggagggggctgcaggttggaactatgcagtgtgctccggccacacatcctgctgggccccctaccctgccccaattcaatcctgccaataaatcctgtcttatttgttcatcctggagaattgaagggaggtcaagttgtttgtcaatgatttgtcagagaacctgttgaaatgtgaattaagaagctaagaaaatatttcttagcaacattttctttttcttttttttttttttcttttgagacagagtctcactctcgtcgcccaggctggaatgcagtggtgcgatctcggctctctgcaacctctgtctcccgggttcaagcgatttcctgcgtcagccccagagtagctggaattacaggcacacaccaccacgcctggctaatttttgtatttttagtagagctggggccaccctggcccggccccgtcttcctccccaaaggtcagactgcaggctgcagggctgtgctggaggagccagctctagctcacccatgcttttgcaacagggtcgggttggaagtcagcacaggtcagtcctgcggaaggttccttcgtgactcatctgtgaagtggggtggttgggagaggtagctgagagaatgcatgagagtcctcggtgcctggcaggaggctggaaggttctagaacactgatggttataagagtgggactgtgagcctgggatcggggggtgtgagacttggatgggagcacaagagtggaaacacagcttctgcacggagcaggcgcagccctcaacaccccgtgcacctgcaccctagggactcttgggtccagatgtgctgtggttttcacaccttcttgggggcaacaggttccaggagccacctgtgggtgccacctgagccacaggctcccaggaaagcagcacagctctcctgcacccagagcttgctgggtggcggaggggaacacagatggttggggaaggcctgaggccagattgggggactctggactggggcagatgaggctcctcagaatcccacctttgaagggaactcagcttataaacacagaggagcaaagttggagggccgggcgtagtggctcacacctgtgatctcagcactttgggaggccaaggaaggtggatcacttgaggccaggagttcgagaccagcctgggcaacatagcaaggccccatctctacaaaaattattattttttaaaaaaattagccaggtgtggtggtgcttgcctatagtcccagctactcgggaggctaaggtgggaggatcgctggagcccaggaatttgaggctgcagtgagctgtgattacaccgttgcactccagcctgggtcacagatcaagaccctgtctcttaaaaataaaagttggagacaagagctggctcacctgaaaggagggattagtaggtaggagggtggatggaggatggatggatgtgtgggtggataggaagatggtattaagttggtgcaaaagtctttgatattactcttaatggctttaataaaaagcttgaaggaagaatgattggttggatagacagagataaatgcatactggaaacaaagataaagataaaacacaagttataccaggccagcaactctattttgttcactgcctttagtcccagcctggcacatagtaggcactcaataaagcctgatttgtagcaaaaaaaaaaaaaaaaa

SEQ ID NO: 10 - protein, 538 aa, Homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 11 - protein, 538 aa, Homo sapiens; identical to SEQ ID NO: 10

SEQ ID NO: 12 - protein, 560 aa, Homo sapiens
MSCRCIFLMKHGERVGWPVGVSMPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 13 - DNA, 2607 bp, Mus musculus
agatagacaccgaagcctccgctggtggccctgtgtttcagtcgcacacagctgtctgcccacttctcctgtggtgtgcctcacggtcacttgcttgtctgaccgcaagtctgcccatccctggggcagccaactggcctcagcccgtgccccaggcgtgccctgtctctgtctggctgccccagccctactgtcttcctctgtgtaggctctgcccagatgcccggctggtcctcagcctcaggactatctcagcagtgactcccctgattctggacttgcacctgactgaactcctgcccacctcaaaccttcacctcccaccaccaccactccgagtcccgctgtgactcccacgcccaggagaccacccaagtgccccagcctaaagaatggctttctgagaaagaccctgaaggagtaggtctgggacacagcatgccccggggcccagtggctgccttactcctgctgattctccatggagcttggagctgcctggacctcacttgctacactgactacctctggaccatcacctgtgtcctggagacacggagccccaaccccagcatactcagtctcacctggcaagatgaatatgaggaacttcaggaccaagagaccttctgcagcctacacaggtctggccacaacaccacacatatatggtacacgtgccatatgcgcttgtctcaattcctgtccgatgaagttttcattgtcaatgtgacggaccagtctggcaacaactcccaagagtgtggcagctttgtcctggctgagagcatcaaaccagctccccccttgaacgtgactgtggccttctcaggacgctatgatatctcctgggactcagcttatgacgaaccctccaactacgtgctgaggggcaagctacaatatgagctgcagtatcggaacctcagagacccctatgctgtgaggccggtgaccaagctgatctcagtggactcaagaaacgtctctcttctccctgaagagttccacaaagattctagctaccagctgcaggtgcgggcagcgcctcagccaggcacttcattcagggggacctggagtgagtggagtgaccccgtcatctttcagacccaggctggggagcccgaggcaggctgggaccctcacatgctgctgctcctggctgtcttgatcattgtcctggttttcatgggtctgaagatccacctgccttggaggctatggaaaaagatatgggcaccagtgcccacccctgagagtttcttccagcccctgtacagggagcacagcgggaacttcaagaaatgggttaatacccctttcacggcctccagcatagagttggtgccacagagttccacaacaacatcagccttacatctgtcattgtatccagccaaggagaagaagttcccggggctgccgggtctggaagagcaactggagtgtgatggaatgtctgagcctggtcactggtgcataatccccttggcagctggccaagcggtctcagcctacagtgaggagagagaccggccatatggtctggtgtccattgacacagtgactgtgggagatgcagagggcctgtgtgtctggccctgtagctgtgaggatgatggctatccagccatgaacctggatgctggccgagagtctggccctaattcagaggatctgctcttggtcacagaccctgcttttctgtcttgcggctgtgtctcaggtagtggtctcaggcttggaggctccccaggcagcctactggacaggttgaggctgtcatttgcaaaggaaggggactggacagcagacccaacctggagaactgggtccccaggagggggctctgagagtgaagcaggttccccccctggtctggacatggacacatttgacagtggctttgcaggttcagactgtggcagccccgtggagactgatgaaggaccccctcgaagctatctccgccagtgggtggtcaggacccctccacctgtggacagtggagcccagagcagctagcatataataaccagctatagtgagaagaggcctctgagcctggcatttacagtgtgaacatgtaggggtgtgtgtgtgtgtgtgtgtgtgtgtgtgtgtgtgtcttgggttgtgtgttagcacatccatgttgggatttggtctgttgctatgtattgtaatgctaaattctctacccaaagttctaggcctacgagtgaattctcatgtttacaaacttgctgtgtaaaccttgttccttaatttaataccattggttaaataaaattggctgcaaccaattactggagggattagaggtagggggcttttgagttacctgtttggagatggagaaggagagaggagagaccaagaggagaaggaggaaggagaggagaggagaggagaggagaggagaggagaggagaggagaggagaggagaggagaggctgccgtgaggggagagggaccatgagcctgtggccaggagaaacagcaagtatctggggtacactggtgaggaggtggccaggccagcagttagaagagtagattaggggtgacctccagtatttgtcaaagccaattaaaataac

SEQ ID NO: 14 - protein, 529 aa, Mus musculus
MPRGPVAALLLLILHGAWSCLDLTCYTDYLWTITCVLETRSPNPSILSLTWQDEYEELQDQETFCSLHRSGHNTTHIWYTCHMRLSQFLSDEVFIVNVTDQSGNNSQECGSFVLAESIKPAPPLNVTVAFSGRYDISWDSAYDEPSNYVLRGKLQYELQYRNLRDPYAVRPVTKLISVDSRNVSLLPEEFHKDSSYQLQVRAAPQPGTSFRGTWSEWSDPVIFQTQAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPRSYLRQWVVRTPPPVDSGAQSS